SEQUENCE LISTING (24 sequences)

SEQ ID NO: 1 - DNA, 2323 bp, Homo sapiens
ggcgagaggaagcagggaggagagtgatttgagtagaaaagaaacacagcattccaggctggccccacctctatattgataagtagccaatgggagcgggtagccctgatccctggccaatggaaactgaggtaggcgggtcatcgcgctggggtctgtagtctgagcgctacccggttgctgctgcccaaggaccgcggagtcggacgcaggcagaccatgtggaccctggtgagctgggtggccttaacagcagggctggtggctggaacgcggtgcccagatggtcagttctgccctgtggcctgctgcctggaccccggaggagccagctacagctgctgccgtccccttctggacaaatggcccacaacactgagcaggcatctgggtggcccctgccaggttgatgcccactgctctgccggccactcctgcatctttaccgtctcagggacttccagttgctgccccttcccagaggccgtggcatgcggggatggccatcactgctgcccacggggcttccactgcagtgcagacgggcgatcctgcttccaaagatcaggtaacaactccgtgggtgccatccagtgccctgatagtcagttcgaatgcccggacttctccacgtgctgtgttatggtcgatggctcctgggggtgctgccccatgccccaggcttcctgctgtgaagacagggtgcactgctgtccgcacggtgccttctgcgacctggttcacacccgctgcatcacacccacgggcacccaccccctggcaaagaagctccctgcccagaggactaacagggcagtggccttgtccagctcggtcatgtgtccggacgcacggtcccggtgccctgatggttctacctgctgtgagctgcccagtgggaagtatggctgctgcccaatgcccaacgccacctgctgctccgatcacctgcactgctgcccccaagacactgtgtgtgacctgatccagagtaagtgcctctccaaggagaacgctaccacggacctcctcactaagctgcctgcgcacacagtgggggatgtgaaatgtgacatggaggtgagctgcccagatggctatacctgctgccgtctacagtcgggggcctggggctgctgcccttttacccaggctgtgtgctgtgaggaccacatacactgctgtcccgcggggtttacgtgtgacacgcagaagggtacctgtgaacaggggccccaccaggtgccctggatggagaaggccccagctcacctcagcctgccagacccacaagccttgaagagagatgtcccctgtgataatgtcagcagctgtccctcctccgatacctgctgccaactcacgtctggggagtggggctgctgtccaatcccagaggctgtctgctgctcggaccaccagcactgctgcccccagggctacacgtgtgtagctgaggggcagtgtcagcgaggaagcgagatcgtggctggactggagaagatgcctgcccgccgggcttccttatcccaccccagagacatcggctgtgaccagcacaccagctgcccggtggggcagacctgctgcccgagcctgggtgggagctgggcctgctgccagttgccccatgctgtgtgctgcgaggatcgccagcactgctgcccggctggctacacctgcaacgtgaaggctcgatcctgcgagaaggaagtggtctctgcccagcctgccaccttcctggcccgtagccctcacgtgggtgtgaaggacgtggagtgtggggaaggacacttctgccatgataaccagacctgctgccgagacaaccgacagggctgggcctgctgtccctaccgccagggcgtctgttgtgctgatcggcgccactgctgtcctgctggcttccgctgcgcagccaggggtaccaagtgtttgcgcagggaggccccgcgctgggacgcccctttgagggacccagccttgagacagctgctgtgagggacagtactgaagactctgcagccctcgggaccccactcggagggtgccctctgctcaggcctccctagcacctccccctaaccaaattctccctggaccccattctgagctccccatcaccatgggaggtggggcctcaatctaaggccttccctgtcagaagggggttgtggcaaaagccacattacaagctgccatcccctccccgtttcagtggaccctgtggccaggtgcttttccctatccacaggggtgtttgtgtgtgtgcgcgtgtgcgtttcaataaagtttgtacactttcaaaaaaaaaaaaaaaaaa

SEQ ID NO: 2 - protein, 593 aa, Homo sapiens
MWTLVSWVALTAGLVAGTRCPDGQFCPVACCLDPGGASYSCCRPLLDKWPTTLSRHLGGPCQVDAHCSAGHSCIFTVSGTSSCCPFPEAVACGDGHHCCPRGFHCSADGRSCFQRSGNNSVGAIQCPDSQFECPDFSTCCVMVDGSWGCCPMPQASCCEDRVHCCPHGAFCDLVHTRCITPTGTHPLAKKLPAQRTNRAVALSSSVMCPDARSRCPDGSTCCELPSGKYGCCPMPNATCCSDHLHCCPQDTVCDLIQSKCLSKENATTDLLTKLPAHTVGDVKCDMEVSCPDGYTCCRLQSGAWGCCPFTQAVCCEDHIHCCPAGFTCDTQKGTCEQGPHQVPWMEKAPAHLSLPDPQALKRDVPCDNVSSCPSSDTCCQLTSGEWGCCPIPEAVCCSDHQHCCPQGYTCVAEGQCQRGSEIVAGLEKMPARRASLSHPRDIGCDQHTSCPVGQTCCPSLGGSWACCQLPHAVCCEDRQHCCPAGYTCNVKARSCEKEVVSAQPATFLARSPHVGVKDVECGEGHFCHDNQTCCRDNRQGWACCPYRQGVCCADRRHCCPAGFRCAARGTKCLRREAPRWDAPLRDPALRQLL

SEQ ID NO: 3 - protein, 16 aa, Homo sapiens
PRWDAPLRDPALRQLL

SEQ ID NO: 4 - protein, 48 aa, Homo sapiens
CCGCGCTGGGACGCCCCTTTGAGGGACCCAGCCTTGAGACAGCTGCTG

SEQ ID NO: 5 - protein, 15 aa, Homo sapiens
HLSLPDPQALKRDVP

SEQ ID NO: 6 - protein, 45 aa, Homo sapiens
CACCTCAGCCTGCCAGACCCACAAGCCTTGAAGAGAGATGTCCCC

SEQ ID NO: 7 - protein, 20 aa, Homo sapiens
RREAPRWDAPLRDPALRQLL

SEQ ID NO: 8 - protein, 60 aa, Homo sapiens
CGCAGGGAGGCCCCGCGCTGGGACGCCCCTTTGAGGGACCCAGCCTTGAGACAGCTGCTG

SEQ ID NO: 9 - protein, 27 aa, Homo sapiens
QGPHQVPWMEKAPAHLSLPDPQALKRD

SEQ ID NO: 10 - DNA, 81 bp, Homo sapiens
caggggccccaccaggtgccctggatggagaaggccccagctcacctcagcctgccagacccacaagccttgaagagagat

SEQ ID NO: 11 - protein, 17 aa, Homo sapiens
MWTLVSWVALTAGLVAG

SEQ ID NO: 12 - DNA, 51 bp, Homo sapiens
atgtggaccctggtgagctgggtggccttaacagcagggctggtggctgga

SEQ ID NO: 13 - protein, 33 aa, Homo sapiens
TRCPDGQFCPVACCLDPGGASYSCCRPLLDKWP

SEQ ID NO: 14 - DNA, 120 bp, Homo sapiens
acgcggtgcccagatggtcagttctgccctgtggcctgctgcctggaccccggaggagccagctacagctgctgccgtccccttctggacaaatggcccacaacactgagcaggcatctg

SEQ ID NO: 15 - protein, 9 aa, Homo sapiens
QRSGNNSVG

SEQ ID NO: 16 - DNA, 27 bp, Homo sapiens
caaagatcaggtaacaactccgtgggt

SEQ ID NO: 17 - protein, 26 aa, Homo sapiens
TPTGTHPLAKKLPAQRTNRAVALSSS

SEQ ID NO: 18 - DNA, 78 bp, Homo sapiens
acacccacgggcacccaccccctggcaaagaagctccctgcccagaggactaacagggcagtggccttgtccagctcg

SEQ ID NO: 19 - protein, 18 aa, Homo sapiens
KENATTDLLTKLPAHTVG

SEQ ID NO: 20 - DNA, 57 bp, Homo sapiens
tccaaggagaacgctaccacggacctcctcactaagctgcctgcgcacacagtgggg

SEQ ID NO: 21 - protein, 24 aa, Homo sapiens
RGSEIVAGLEKMPARRASLSHPRD

SEQ ID NO: 22 - DNA, 72 bp, Homo sapiens
cgaggaagcgagatcgtggctggactggagaagatgcctgcccgccgggcttccttatcccaccccagagac

SEQ ID NO: 23 - protein, 21 aa, Homo sapiens
KEVVSAQPATFLARSPHVGVK

SEQ ID NO: 24 - DNA, 63 bp, Homo sapiens
aaggaagtggtctctgcccagcctgccaccttcctggcccgtagccctcacgtgggtgtgaag

(19) United States
(12) Patent Application Publication
(10) Pub. No.: US 2014/0356321 A1
(43) Pub. Date: Dec. 4, 2014

(54) MATERIALS AND METHODS FOR TREATMENT OF LIVER CANCER

(71) Applicant: THE UNIVERSITY OF HONG KONG, Hong Kong (CN)

(72) Inventors: Siu Tim Cheung, Hong Kong (CN); Sheung-Tat Fan, Hong Kong (CN); Phyllis Fung Yi Cheung, HONG KONG (CN)

(73) Assignee: THE UNIVERSITY OF HONG KONG, Hong Kong (CN)

(21) Appl. No.: 14/292,045

(22) Filed: May 30, 2014

Related U.S. Application Data
(60) Provisional application No. 61/829,027, filed on May 30, 2013.

(51) Int. Cl. C07K 16/22; A61K 45/06; C12N 15/113

(52) U.S. Cl. CPC C07K 16/22; C12N 15/113; A61K 45/06; A61K 2039/505
USPC 424/85.2; 424/85.4; 424/145.1; 424/158.1; 424/93.71; 514/44 A; 514/54

(57) ABSTRACT

Granulin-epithelin precursor (GEP), a pluripotent growth factor, is a hepatic oncofetal protein defining a cancer stem cell (CSC) population in liver cancer. The present invention provides the use of GEP inhibitors (including anti-GEP antibody) for inhibiting immune evasion by liver cancer cells and for eliminating liver cancer stem cells.

### CROSS-REFERENCE TO RELATED APPLICATION

This application claims the benefit of U.S. provisional application Ser. No. 61/829,027, filed May 30, 2013, which is herein incorporated by reference in its entirety.

### BACKGROUND OF THE INVENTION

Liver cancer is the third leading cancer killer in the world, with more than half a million individuals dying globally each year. In China, liver cancer is the second major cause of cancer death. Surgical resection, in the form of a partial hepatectomy or a liver transplant, is the mainstay of curative treatment. Nonetheless, cancer recurrence is still common after curative surgery. In addition, liver cancer is frequently diagnosed at an advanced stage, which precludes curative treatment. No effective therapeutic option exists for the treatment of the majority of liver cancer patients.

Granulin-epithelin precursor (GEP) is a pluripotent growth factor regulating fetal development, tissue repair and tumorigenesis in various cancers. GEP is over-expressed in more than 70% of human hepatocellular carcinoma (HCC). GEP expression has been shown to associate with aggressive HCC features. Functional studies demonstrate that GEP plays a role in regulating HCC cell proliferation, invasion, tumorigenicity, and chemoresistance. Moreover, neutralization of GEP could inhibit the growth of established HCC in mouse model.

Cancer stem cells (CSCs) are considered as the “root” of cancers. CSCs are not only responsible for tumor initiation and progression, but also endowed with stem cell properties, including self-renewal, differentiation capacity and chemoresistance. Although existing therapies can initially eliminate the tumor bulk, stem cell properties of CSCs enable them to survive and repopulate the tumor, resulting in disease relapse. Thus, CSC eradication may be the key for curing aggressive malignancies including HCC.

The immune system has the ability to identify and eliminate tumor cells before they develop into malignancy. NK cells are the major component of the innate immune system and represent the first line of defense against tumors. Anti-tumor cytotoxicity of NK cells is mediated by direct lysis or induction of IFN-γ. However, it was reported that cytotoxicity of NK cells was impaired in HCC patients and the reduced activities of NK cells are associated with HCC progression.

NK cytotoxicity is regulated through integrated signaling from their cell surface receptors that interact with ligands expressed on target cells. NKG2D (natural killer group 2, member D) is a stimulatory receptor expressed on the surface of all NK cells and its recognition is crucial for tumor immunosurveillance. MHC class I chain-related molecule A (MICA), a ligand of human NKG2D receptor, is frequently expressed in tumors, but not in normal tissues. Engagement of MICA and NKG2D strongly activates NK cells, enhancing their cytotoxicity and cytokine production. Thus, the NKG2D-MICA pathway is an important mechanism by which host immune system recognizes and eliminates tumor cells. CD94/NKG2A is an inhibitory receptor that controls the activity of human NK cells following interactions with the non-classic class I human leukocyte antigen E (HLA-E) on target cells. HLA-E is ubiquitously expressed by nearly all cells in the body, but is over-expressed by tumor cells and is likely to protect the tumor cells from NK cytotoxic activity through inhibition via interactions with the CD94/NKG2A receptor.

Immune evasion is one of the important properties of CSCs that enable them to survive better in the host. Recently, ABCB5+ malignant melanoma initiating cells were found to possess the capacity to modulate anti-tumor immunity by preferentially inhibiting IL-2-dependent T-cell activation and to support induction of regulatory T cells. Moreover, CD200+ CSC cells were found to suppress anti-tumor immunity by down-regulating the expression of Th1 cytokines and co-stimulatory molecules in ovarian cancer, melanoma and leukemia. However, evaluation of CSCs in the context of host immune responses has largely been disregarded in HCC. Therapeutic regimes that inhibit tumor cells from escaping from immunosurveillance can be used to treat hepatic CSCs.

### BRIEF SUMMARY OF THE INVENTION

The present invention provides a method for suppressing immune evasion and for eradicating GEP-expressing CSCs in hepatocellular carcinoma (HCC). The present invention is based on the surprising discovery that granulin-epithelin precursor (GEP) expression is associated with the immune evasion ability of human liver cancer; GEP is associated with the natural killer (NK) cell activity in human liver cancer; and anti-GEP antibody treatment enhances the NK cell cytotoxic activities against liver cancer cells.

In one embodiment, the present invention provides a method for treating liver cancer, wherein the method comprises administering to a subject in need of such treatment an agent that inhibits the expression or activity of granulin-epithelin precursor (GEP) and, optionally, NK cells and/or an agent that promotes natural killer (NK) cell activity.

In one embodiment, the agent that promotes NK cell activity enhances cytotoxicity of NK cells.

In one specific embodiment, the present invention provides a method for treating liver cancer, wherein the method comprises administering to a subject in need of such treatment an antibody that binds to granulin-epithelin precursor (GEP) and, optionally, NK cells and/or an agent that promotes natural killer (NK) cell activity.

In one embodiment, the subject has undergone partial or total hepatectomy and/or liver transplantation, and the present invention can be used to prevent or reduce the likelihood of liver tumor or cancer recurrence.

### BRIEF DESCRIPTION OF THE DRAWINGS

FIG. 1 shows that GEP expression of HCC cells modulates anti-tumor cytotoxic activity of immune cells. (A) GEP modulation by transfection in HCC cell lines. GEP protein and transcript levels were significantly suppressed in Hep3B (high endogenous GEP cell line, left panel); while significantly over-expressed in HepG2 (low endogenous GEP cell line, right panel). GEP protein level was expressed as MFI (Mean fluorescence intensity), after subtracting that of isotype control. (B) GEP suppression increased, while over-expression decreased PBMC cytotoxicity against HCC cells. HCC cells (target cells) were co-cultured with PBMC (effector cells) at effector/target ratio (E:T ratio) of 25:1 for 5 h. *p<0.05 when compared GEP transfectants with control cells. (C) Depletion of NK cells markedly abolished the GEP-mediated cell cytotoxicity. CD56+ NK cells were depleted from PBMC by magnetic sorting. NK cells-depleted PBMC were then co-cultured with HCC cells at E:T ratio of 25:1 for 5 h. (D) GEP suppression increased, while over-expression decreased NK cytotoxicity against HCC cells. *p<0.05, **p<0.01 when compared with control cells at respective E:T ratio. NK cells were also isolated from liver cancer patients and cytotoxicity against liver cancer cells analyzed, and (E) the cells showed enhanced cytotoxicity in liver cancer cells with GEP suppression, and (F) decreased cytotoxicity in liver cancer cells with GEP over-expression.

FIG. 2 shows that GEP differentially regulates the expression of MICA and HLA-E of HCC cells and NKG2d on NK cells. (A) GEP suppression up-regulated, while over-expression down-regulated surface MICA on HCC cells. MFI: mean fluorescence intensity (after subtracting that of isotype control). (B) GEP suppression down-regulated, while over-expression up-regulated surface HLA-E on HCC cells. MFI: mean fluorescence intensity (after subtracting that of isotype control). (C) GEP suppression reduced, while over-expression augmented soluble MICA release from HCC cells. (D) Co-culture of GEP-suppressed HCC cells increased, while GEP-over-expressed HCC cells decreased NKG2D expression on NK cell surface. Dotted line in histogram: isotype control staining MFI: mean fluorescence intensity (after subtracting that of isotype control). * p<0.05, **p<0.01, *** p<0.001 when compared with control cells.

FIG. 3 shows that GEP blockage by anti-GEP antibody A23 modulates the expression of MICA and HLA-E of HCC cells. Hep3B cells were treated with anti-GEP antibody (A23), mouse IgG isotype (IgG) (50 μg/ml) or without antibody (control) in serum-starved condition (1% FBS) for 24 h. (A) A23 significantly reduced GEP expression of Hep3B cells. A23 induced cell surface MICA expression, while suppressed soluble MICA release and cell surface HLA-E expression of Hep3B cells. Dotted line: isotype control staining * p<0.05, ** p<0.01 when compared with control. MFI: mean fluorescence intensity (after subtracting that of isotype control). (B) Orthotopic tumors of Hep3B cells were established in nude mice, which were then subjected to saline (control) or 10 mg/kg anti-GEP antibody A23 (A23 treatment) twice weekly for six weeks. Tumors were stained by immunohistochemistry for GEP and MICA proteins.

FIG. 4 shows that GEP blockage by anti-GEP antibody A23 enhances NK cell cytotoxicity against HCC cells via MICA. Hep3B and HepG2 cells were treated with anti-GEP monoclonal antibody (A23), mouse IgG isotype control (IgG) (50 μg/ml) or without antibody (control) in serum-starved condition (1% FBS) for 24 h prior to co-culture with PBMC at E:T ratio of 25:1 for 5 h. For A23 treatment, cells were co-cultured with PBMC with or without neutralizing MICA antibody (anti-MICA) or mouse IgG2a isotype control (IgG2a) (2 μg/ml) for 5 h. (A) GEP blockage by anti-GEP antibody A23 enhances PBMC cytotoxicity against HCC cells. The cytotoxicity is suppressed by anti-MICA neutralizing antibody. *P<0.05 when compared with control; # p<0.05 between groups denoted by horizontal lines. (B) A23-enhanced NK cytotoxicity against Hep3B and HepG2 cells was suppressed by anti-MICA neutralizing antibody. * p<0.05, ** p<0.01, *** p<0.001 when compared with control; # p<0.05, ## p<0.001 when compared with A23 alone treatment at respective E:T ratio.

FIG. 5 shows the expression patterns of GEP, MICA and HLA-E in HCC clinical specimens. (A) GEP, MICA and HLA-E mRNA levels were up-regulated in HCC compared with the paralleled adjacent non-tumor liver tissues and normal livers from healthy individuals (p=0.000, p=0.000 and p=0.089, respectively). (B) Immunohistochemical staining showed stronger expression of GEP, MICA and HLA-E proteins in HCC than the paralleled adjacent non-tumor liver tissues. (C) Serum GEP (sGEP) and MICA (sMICA) levels were significantly higher in HCC patients than those of healthy individuals (p=0.010 and p<0.001, respectively). (D) Liver tissues were digested into disaggregated cells, and assessed for the expression of surface MICA, HLA-E and GEP. Expressions of surface MICA and GEP were mutually exclusive (left panel). Surface HLA-E co-expressed with GEP (right panel). Table summarizes the mean percentage of positive cells±SD from 3 HCC clinical specimens respectively.

FIG. 6 shows antibody-dependent cellular cytotoxicity mediated by anti-GEP monoclonal antibody A23. A23 mediated ADCC against HCC cells dose-dependently. Hep3B or HepG2 cells labeled with CFSE were incubated with or without anti-GEP antibody A23 (A23) or mouse isotype control (migG) of the indicated concentrations of the antibodies for 30 min prior to co-culture with or without PBMC at E/T ratio of 25:1 for 5 h. * P<0.05, * P<0.01 when compared with control cells without antibody. (B) Depletion of CD56+ NK cells from human PBMC markedly abolished A23-mediated ADCC. Hep3B or HepG2 cells labeled with CFSE were incubated with or without anti-GEP antibody A23 of indicated concentrations for 30 min prior to co-culture with PBMC or NK cell-depleted PBMC at E/T ratio of 25:1 for 5 h. * P<0.05, ** P<0.01 when compared with control cells without antibody. (C) A23-mediated ADCC against HCC cells was dependent on NK cells. CD56+ NK cells were isolated and cultured with HCC cells for 5 hr at indicated E/T ratio. *P<0.05, **P<0.01, ***P<0.001 when compared with control cells without antibody.

FIG. 7 shows association of serum GEP and MICA levels with recurrence-free survival in HCC patients. Kaplan-Meier recurrence-free survival plot according to serum GEP (sGEP) and MICA (sMICA) levels. Patients were segregated into low expression group (low in both sGEP and sMICA) and high expression group (either one or both high in sGEP and sMICA) with the Youden index maximized to determine the optimal cutoff value. There were 25 patients in the low expression group (median recurrence-free survival, 51.2 months) and 55 patients in the high expression group (median recurrence-free survival, 12.8 months). (A) Patients with high expression levels of sGEP and/or sMICA (sGEP/MICA high) were found to have poor recurrence-free survival (log-rank test, p=0.042). When the patients were segregated into (B) early and (C) advanced tumor stages, patients with high sGEP and/or sMICA (sGEP/MICA high) were also shown to have poor recurrence-free survival (log-rank test, p=0.032).

FIG. 8 shows that GEP does not regulate the expression of HLA-G and HLA-A/B/C of HCC cells. (A) Hep3B cells (high endogenous GEP cell line) were stably transfected for GEP suppression by shRNA, while (B) HepG2 cells (low endogenous GEP cell line) were stably transfected for over-expression by full-length GEP cDNA. Surface HLA-G and HLA-A/B/C expression on the HCC cells and their GEP transfectants was measured by surface immunofluorescence staining and flow cytometry. Dotted line: isotype control staining.

### BRIEF DESCRIPTION OF THE SEQUENCES

SEQ ID NO:1 is a cDNA sequence of a granulin-epithelin precursor (GEP).

SEQ ID NO:2 is the amino acid sequence of a granulin-epithelin precursor.

SEQ ID NO:3 is the amino acid sequence of part of GEP (GEP amino acids 578-593).

SEQ ID NO:4 is the nucleic acid sequence encoding amino acids 578-593 of GEP.

SEQ ID NO:5 is the amino acid sequence of part of GEP (GEP amino acids 351-365).

SEQ ID NO:6 is the nucleic acid sequence encoding amino acids 351-365 of GEP.

SEQ ID NO:7 is the amino acid sequence of part of GEP (GEP amino acids 574-593).

SEQ ID NO: 8 is the nucleic acid sequence encoding amino acids 574-593 of GEP.

SEQ ID NO:9 is the amino acid sequence of part of GEP (GEP amino acids 337-363).

SEQ ID NO:10 is the nucleic acid sequence encoding amino acids 337-363 of GEP.

SEQ ID NO:11 is the amino acid sequence of part of GEP signaling peptide (GEP amino acids 1-17).

SEQ ID NO:12 is the nucleic acid sequence encoding amino acids 1-17 of GEP.

SEQ ID NO:13 is the amino acid sequence of part of the GEP linker (GEP amino acids 18-57).

SEQ ID NO:14 is the nucleic acid sequence encoding amino acids 18-57 of GEP.

SEQ ID NO:15 is the amino acid sequence of part of the GEP linker (GEP amino acids 114-122).

SEQ ID NO:16 is the nucleic acid sequence encoding amino acids 114-122 of GEP.

SEQ ID NO:17 is the amino acid sequence of part of the GEP linker (GEP amino acids 180-205).

SEQ ID NO:18 is the nucleic acid sequence encoding amino acids 180-205 of GEP.

SEQ ID NO:19 is the amino acid sequence of part of the GEP linker (GEP amino acids 262-280).

SEQ ID NO:20 is the nucleic acid sequence encoding amino acids 262-280 of GEP.

SEQ ID NO:21 is the amino acid sequence of part of the GEP linker (GEP amino acids 418-441).

SEQ ID NO:22 is the nucleic acid sequence encoding amino acids 418-441 of GEP.

SEQ ID NO:23 is the amino acid sequence of part of the GEP linker (GEP amino acids 497-517).

SEQ ID NO:24 is the nucleic acid sequence encoding amino acids 497-517 of GEP.

### DETAILED DISCLOSURE OF THE INVENTION

The present invention provides a method for suppressing immune evasion and for eradicating the GEP-expressing CSCs in hepatocellular carcinoma (HCC). The present invention is based on the surprising discovery that granulin-epithelin precursor (GEP) expression is associated with the immune evasion ability of human liver cancer; GEP is associated with the natural killer (NK) cell activity in human liver cancer; and anti-GEP antibody treatment enhances the NK cell cytotoxic activities against liver cancer cells.

### Effect of GEP on Immune Evasion and Cancer Stem Cell Population

The present invention shows that granulin-epithelin precursor (GEP) rendered hepatocellular carcinoma (HCC) cells resistant to cytotoxic activity of NK cells, an important mechanism of anti-tumor responses. The alteration in immune response induced by GEP would cause an imbalance in the host's immune responses, allowing the subsequent growth of a tumor. Thus, GEP provides a selective advantage to cancer stem cells (CSCs) by enabling them to proliferate in hosts.

GEP blockage by monoclonal antibody A23 significantly suppresses GEP level in HCC cells, and the GEP-mediated regulation on MHC class I chain-related molecule A (MICA) and HLA-E expression on HCC cells could be reversed. As a result, the HCC cells can be sensitized to NK cytotoxic activity. In addition, the present invention shows that anti-GEP monoclonal antibody A23 induces NK cell-mediated ADCC against HCC cells, and therefore further amplifies the anti-tumor response of the antibody. Therefore, targeting antibody that binds specific to GEP (such as A23 antibody) can be used for suppressing immune evasion and eradicating the GEP-expressing CSCs in HCC.

GEP defines a hepatic CSC population with greater chemoresistance, self-renewal and tumor-initiating ability. The present inventors discovered that GEP conferred the CSCs' ability to escape from NK immunosurveillance, giving a selective advantage to the GEP⁺ cells and enabling them to survive and proliferate in the host.

As a hallmark of tumor progression and recurrence, tumors have developed diverse mechanisms to evade the immune system. It is postulated that tumor cells have evolved to down-regulate their surface MICA by proteolytic shedding, therefore reducing their susceptibility to NKG2D-mediated NK cytotoxicity. Also, the soluble MICA (sMICA) released from tumor cell surface can not only block the NKG2D-binding site for other NKG2D ligands, but also suppress NKG2D expression on NK cells. Production of sMICA therefore represents one of the mechanisms in tumor immune evasion. It was reported that sMICA were frequently elevated in advanced HCC patients and such elevation was associated with down-regulated NKG2D expression and impaired NK cell activation in HCC. Significance of sMICA as predictive and prognostic marker has been reported in HBV-induced HCC and advanced HCC.

The present invention shows that GEP suppression significantly up-regulated, while GEP over-expression reduced surface MICA level on HCC cells. GEP-expressing cells might therefore evade NK cytotoxicity by reducing surface MICA level and become less susceptible to NK cell responses. In addition, sMICA level decreased significantly in the culture supernatant of GEP-suppressed HCC cells, but increased significantly when GEP was over-expressed. The results show that GEP could confer HCC cells immune evasion ability by promoting shedding of MICA from cell surface and promote the release of sMICA which disturbs the NKG2D-mediated NK cytotoxicity.

Up-regulation of HLA-E, a ligand for the NK inhibitory immunoreceptor CD94/NKG2A9, has been suggested to represent a mechanism of tumor escape from NK cell immunosurveillance in ovarian cancer and melanoma. It was previously reported that HLA-E mRNA was up-regulated in HCC specimens in comparison with their adjacent normal counterpart. Also, HLA-E polymorphism was also associated with risk of hepatitis B or HCC.

Nevertheless, before the present invention, the role of HLA-E in HCC pathogenesis has not been reported. In addition, it was not elucidated that HCC cells over-express HLA-E.

The present invention shows that GEP expression up-regulated HCC cells' surface expression of HLA-E, which interacts with NK cell inhibitory immunoreceptor CD94/NKG2A and suppresses NK cell activity.

HLA-E could play a role in HCC pathogenesis by promoting the immune evasion ability of GEP-expressing CSCs. GEP blockage using anti-GEP monoclonal antibody A23 could significantly suppress GEP level and sensitize the GEP⁺ cells to NK cytotoxic activity. The results show that antibodies that bind to GEP have antitumor effect against HCC cells once NK cells are efficiently activated during A23 treatment. The combination therapy of anti-HCC molecular targeted therapy and immunotherapy targeting activation of NK cells could improve the antitumor effect against unresectable HCC and the prognosis of patients with HCC. Also, as the present inventors have shown that GEP expression would induce shedding of MICA from HCC cell surface and increase soluble MICA, patients with higher soluble MICA level in their serum would be more responsive to the anti-GEP monoclonal antibody treatment. The present invention can be used to define criteria to select patients who are potentially responsive to anti-GEP antibody treatment.

In addition to immune escape, another immunologic mechanism known to play a role in the anti-tumor activity of antibodies is their ability to induce antibody-dependent cell-mediated cytotoxic activity (ADCC) mediated by cytotoxic immune cells (such as NK cells) to lyse the tumor cells. ADCC is an innate immune effector mechanism in which antibodies evoke tumor cell death when antibodies bind to antigen on tumor cells and the antibody Fc domains engage Fc receptors on the surface of immune effector cells.

The present invention shows that anti-GEP monoclonal antibody A23 could induce NK cell-mediated ADCC against HCC cells. Therefore, the anti-tumor effect of anti-GEP monoclonal antibody A23 can be further amplified by inducing destruction of the A23-coated HCC cells by NK cells. Targeting GEP by antibody A23 represents a novel therapeutic tool for suppressing immune evasion and for eradicating the GEP-expressing CSCs in HCC.

### Liver Cancer or Tumor Therapy

In one embodiment, the present invention provides a method for treating liver cancer, wherein the method comprises administering to a subject in need of such treatment an effective amount of an agent that inhibits the expression or activity of granulin-epithelin precursor (GEP) and, optionally, natural killer (NK) cells and/or an agent that promotes NK cell activity.

In one embodiment, the agent that promotes NK cell activity enhances cytotoxicity of NK cells.

In one specific embodiment, the present invention provides a method for treating liver cancer, wherein the method comprises administering to a subject in need of such treatment an antibody that binds to granulin-epithelin precursor (GEP) and, optionally, NK cells and/or an agent that promotes natural killer (NK) cell activity.

In another specific embodiment, the present invention provides a method for treating liver cancer, wherein the method comprises administering to a subject in need of such treatment a GEP antisense polynucleotide and, optionally, NK cells and/or an agent that promotes natural killer (NK) cell activity. In one specific embodiment, the GEP antisense polynucleotide is administered to a liver tumor of the subject.

In one embodiment, the agent that inhibits the expression or activity of granulin-epithelin precursor (GEP) is administered before, during, or after NK cells and/or an agent that promotes NK cell activity. In one embodiment, the method comprises administering a therapeutic composition comprising an agent that inhibits the expression or activity of granulin-epithelin precursor (GEP), NK cells, and/or an agent that promotes NK cell activity.

In another embodiment, the present invention provides a method of treating liver cancer comprising administering to a subject in need of such treatment an effective amount of an agent that increases MHC class I chain-related molecule A (MICA) expression on liver cancer cells. Such agents can be agents that inhibit the activity of GEP, thereby causing an increase in MICA expression on liver cancer cells.

In yet another embodiment, the present invention provides a method of treating liver cancer comprising administering to a subject that has liver cancer an effective amount of an agent that promotes natural killer (NK) cell activity and/or enhances the cytotoxicity of NK cells, such as GEP inhibiting agents.

The term “treatment” or any grammatical variation thereof (e.g., treat, treating, and treatment etc.), as used herein, includes but is not limited to, ameliorating or alleviating a symptom of a disease or condition, reducing, suppressing, inhibiting, lessening, or affecting the progression, severity, and/or scope of a condition.

The term “prevention” or any grammatical variation thereof (e.g., prevent, preventing, and prevention etc.), as used herein, includes but is not limited to, reducing the likelihood of developing a disease, delaying the onset of symptoms, preventing relapse to a disease, increasing latency between symptomatic episodes, or a combination thereof. Prevention, as used herein, does not require the complete absence of symptoms.

The term “effective amount,” as used herein, refers to an amount that is capable of treating or ameliorating a disease or condition or otherwise capable of producing an intended therapeutic effect.

The term “subject,” as used herein, describes an organism, including mammals such as primates, to which treatment with the compositions according to the subject invention can be provided. Mammalian species that can benefit from the disclosed methods of treatment include, but are not limited to, apes, chimpanzees, orangutans, humans, monkeys; and other animals such as dogs, cats, horses, cattle, pigs, sheep, goats, chickens, mice, rats, guinea pigs, and hamsters. In one embodiment, the subject has, or is diagnosed with, liver tumor or cancer. In one embodiment, the subject had underwent partial or total hepatectomy and/or liver transplantation.

The term “partial hepatectomy,” refers to the surgical resction of less than the entire, but more than 5% (including, any percent higher than 5%, including but not limited to, more than 10%, 15%, 20%, 25%, 30%, 35%, 40%, 45%, 50%, 55%, 60%, 65%, 70%, 75%, 80%, 85%, 90%, or 95%) of the liver.

In one embodiment, the present invention can be used to elimitate or reduce liver cancer stem cells. In one embodiment, the administration of one or more GEP inhibitors (such as, anti-GEP antibody) to a subject who had surgical resection of a primary liver tumor can be used to prevent the recurrence of liver tumor or cancer. In one embodiment, the administration of one or more GEP inhibitors (such as, anti-GEP antibody or aptamer) to a subject who had surgical resection of a primary liver tumor can be used to treat liver tumor or cancer.

In one embodiment, one or more GEP inhibitors (such as, anti-GEP antibody or aptamer) to a subject who had partial hepatectomy or liver transplantation. In one embodiment, the present invention can be used to prevent or reduce the likelihood of liver tumor or cancer recurrence.

In one embodiment, the subject has undergone surgical resection of an entire primary liver tumor. In one embodiment, the subject has undergone surgical resection of part of a primary liver tumor.

In one embodiment, one or more GEP inhibitors are administered before or after the subject receives surgical resection of the liver tumor.

The present application incorporates by reference WO2005/106019, WO2008/064570 and WO2011/140828 in their entity. In certain embodiments, the present invention does not encompass the treatment of liver cancer described in WO2005/106019, WO2008/064570 and WO2011/140828.

### GEP Inhibitors

GEP inhibitors useful according to the present invention include, but are not limited to, agents that inhibit GEP activity; and agents that reduce or inhibit the expression of GEP, such as agents that inhibit the transcription and/or translation of GEP.

Agents that inhibit GEP activity include, but are not limited to, anti-GEP antibodies, aptamers, GEP binding partners, and small molecule inhibitors of GEP.

In one embodiment, the GEP inhibitor is an antibody, aptamer, or binding partner that binds to GEP. In a specific embodiment, the GEP inhibitor is an antibody, aptamer, or binding partner that binds specifically to GEP. In a further specific embodiment, the GEP inhibitor is an antibody, aptamer, or binding partner that binds specifically to human GEP.

In one embodiment, the cDNA sequence of a GEP useful according to the present invention is SEQ ID NO. 1. In one embodiment, the GEP protein useful according to the present invention comprises an amino acid sequence of SEQ ID NO. 2.

In a further specific embodiment, the GEP inhibitor is an antibody, aptamer, or binding partner that binds specifically to a GEP of SEQ ID NO:2.

Embodiments of anti-GEP antibodies are also described in WO2008/064570, which is herein incorporated by reference in its entirety.

In one specific embodiment, the present invention provides a method for treating liver cancer, wherein the method comprises administering, to a subject that has liver cancer, a composition comprising an antibody that binds to granulin-epithelin precursor (GEP), and, optionally, NK cells and/or an agent that promotes natural killer (NK) cell activity.

In certain embodiments, the GEP inhibitor is an antibody, aptamer, or binding partner that binds specifically to a GEP protein of non-human animal species including, but not limited to, dogs, cats, horses, pigs, sheep, goats, chickens, mice, rats, and guinea pigs. The skilled artisan can readily make antibodies, aptamers, or binding partners that specifically bind to GEP proteins that are publically known. In another embodiment, the GEP inhibitor is a fusion construct comprising the antibody, aptamer, or binding partner that binds specifically to a GEP protein (such as human GEP).

In one embodiment, GEP-specific antibodies can be generated by immunizing BALB/c mice or New Zealand white rabbits with GEP specific peptide sequences, as described in WO2008/064570, which is herein incorporated by reference in its entirety.

“Specific binding” or “specificity” refers to the ability of a protein to detectably bind an epitope presented on a protein or polypeptide molecule of interest, while having relatively little detectable reactivity with other proteins or structures. Specificity can be relatively determined by binding or competitive binding assays, using, e.g., Biacore instruments. Specificity can be exhibited by, e.g., an about 10:1, about 20:1, about 50:1, about 100:1, 10.000:1 or greater ratio of affinity/avidity in binding to the specific target molecule versus nonspecific binding to other irrelevant molecules.

Anti-GEP antibodies of the present invention can be in any of a variety of forms, including intact immunoglobulin molecules, fragments of immunoglobulin molecules such as Fv, Fab and similar fragments; multimers of immunoglobulin molecules (e.g., diabodies, triabodies, and bi-specific and tri-specific antibodies, as are known in the art; see, e.g., Hudson and Kortt, J. Immunol. Methods 231:177 189, 1999); fusion constructs containing an antibody or antibody fragment; and human or humanized immunoglobulin molecules or fragments thereof.

Antibodies within the scope of the invention can be of any isotype, including IgG, IgA, IgE, IgD, and IgM. IgG isotype antibodies can be further subdivided into IgG1, IgG2, IgG3, and IgG4 subtypes. IgA antibodies can be further subdivided into IgA1 and IgA2 subtypes.

Antibodies of the present invention include polyclonal and monoclonal antibodies. The term “monoclonal antibody,” as used herein, refers to an antibody or antibody fragment obtained from a substantially homogeneous population of antibodies or antibody fragments (i.e. the individual antibodies within the population are identical except for possible naturally occurring mutations that may be present in a small subset of the antibody molecules).

A monoclonal antibody composition is typically composed of antibodies produced by clones of a single cell called a hybridoma that secretes (produces) only one type of antibody molecule. The hybridoma cell is formed by fusing an antibody-producing cell and a myeloma or other self-perpetuating cell line. Such antibodies were first described by Kohler and Milstein, Nature, 1975, 256:495-497, the disclosure of which is herein incorporated by reference. An exemplary hybridoma technology is described by Niman et al., Proc. Natl. Acad. Sci. U.S.A., 1983, 80:4949-4953. Other methods of producing monoclonal antibodies, a hybridoma cell, or a hybridoma cell culture are also well known. See e.g., Antibodies: A Laboratory Manual, Harlow et al., Cold Spring Harbor Laboratory, 1988; or the method of isolating monoclonal antibodies from an immunological repertoise as described by Sasatry, et al., Proc. Natl. Acad. Sci. USA, 1989, 86:5728-5732; and Huse et al., Science, 1981, 246:1275-1281. The references cited are hereby incorporated herein by reference.

In an embodiment, the GEP inhibitor is a GEP antisense polynucleotide. In an embodiment, the GEP inhibitor is an antisense polynucleotide that targets human GEP mRNA. In some embodiments, the GEP antisense polynucleotides target GEP mRNAs of non-human animals including, but not limited to, dogs, cats, horses, pigs, sheep, goats, chickens, mice, rats, and guinea pigs. The skilled artisan would readily appreciate that the antisense polynucleotides can be designed to target any GEP mRNAs publically known.

In some embodiments, the GEP inhibitor is a siRNA or shRNA having a sequence sufficiently complementary to a target GEP mRNA sequence to direct target-specific RNA interference (RNAi). In some embodiments, the GEP inhibitor is siRNA or shRNA having a sequence sufficiently complementary to a target human GEP mRNA sequence to direct target-specific RNA interference.

Examples of antisense polynucleotides include, but are not limited to, single-stranded DNAs and RNAs that bind to complementary target GEP mRNA and inhibit translation and/or induce RNaseH-mediated degradation of the target transcript; siRNA or shRNA oligonucleotides that target or mediate GEP mRNA degradation; ribozymes that cleave GEP mRNA transcripts; and nucleic acid aptamers and decoys, which are non-naturally occurring oligonucleotides that bind to and block GEP protein targets in a manner analogous to small molecule drugs.

The term “nucleotide” refers to a nucleoside having one or more phosphate groups joined in ester linkages to the sugar moiety. Exemplary nucleotides include nucleoside monophosphates, diphosphates and triphosphates. The terms “polynucleotide” and “nucleic acid molecule” are used interchangeably herein and refer to a polymer of nucleotides joined together by a phosphodiester linkage between 5′ and 3′ carbon atoms. The terms “nucleic acid” or “nucleic acid sequence” encompass an oligonucleotide, nucleotide, polynucleotide, or a fragment of any of these, DNA or RNA of genomic or synthetic origin, which may be single-stranded or double-stranded and may represent a sense or antisense strand, peptide nucleic acid (PNA), or any DNA-like or RNA-like material, natural or synthetic in origin. As will be understood by those of skill in the art, when the nucleic acid is RNA, the deoxynucleotides A, G, C, and T are replaced by ribonucleotides A, G, C, and U, respectively.

As used herein, the term “RNA” or “RNA molecule” or “ribonucleic acid molecule” refers generally to a polymer of ribonucleotides. The term “DNA” or “DNA molecule” or deoxyribonucleic acid molecule” refers generally to a polymer of deoxyribonucleotides. DNA and RNA molecules can be synthesized naturally (e.g., by DNA replication or transcription of DNA, respectively). RNA molecules can be post-transcriptionally modified. DNA and RNA molecules can also be chemically synthesized. DNA and RNA molecules can be single-stranded (i.e., ssRNA and ssDNA, respectively) or multi-stranded (e.g., double stranded, i.e., dsRNA and dsDNA, respectively). Based on the nature of the invention, however, the term “RNA” or “RNA molecule” or “ribonucleic acid molecule” can also refer to a polymer comprising primarily (i.e., greater than 80% or, preferably greater than 90%) ribonucleotides but optionally including at least one non-ribonucleotide molecule, for example, at least one deoxyribonucleotide and/or at least one nucleotide analog.

As used herein, the term “nucleotide analog,” also referred to herein as an “altered nucleotide” or “modified nucleotide,” refers to a non-standard nucleotide, including non-naturally occurring ribonucleotides or deoxyribonucleotides. Preferred nucleotide analogs are modified at any position so as to alter certain chemical properties of the nucleotide yet retain the ability of the nucleotide analog to perform its intended function.

As used herein, the term “RNA interference” (“RNAi”) refers to a selective intracellular degradation of RNA. RNAi occurs in cells naturally to remove foreign RNAs (e.g., viral RNAs). Natural RNAi proceeds via fragments cleaved from free dsRNA which direct the degradative mechanism to other similar RNA sequences. Alternatively, RNAi can be initiated by the hand of man, for example, to silence the expression of endogenous target genes.

As used herein, the term “small interfering RNA” (“siRNA”) (also referred to in the art as “short interfering RNAs”) refers to an RNA (or RNA analog) comprising between about 10-50 nucleotides (or nucleotide analogs) which is capable of directing or mediating RNA interference.

As used herein, a siRNA having a “sequence sufficiently complementary to a target mRNA sequence to direct target-specific RNA interference (RNAi)” means that the siRNA has a sequence sufficient to trigger the destruction of the target mRNA (e.g., GEP mRNA) by the RNAi machinery or process. “mRNA” or “messenger RNA” or “transcript” is single-stranded RNA that specifies the amino acid sequence of one or more polypeptides. This information is translated during protein synthesis when ribosomes bind to the mRNA.

The present invention also contemplates vectors (e.g., viral vectors) and expression constructs comprising the nucleic acid molecules useful for inhibiting GEP expression and/or activity. In an embodiment, the vector comprises a siRNA that targets GEP mRNA. In another embodiment, the vector comprises a nucleic acid molecule encoding an anti-GEP A antibody.

As used herein, the term “expression construct” refers to a combination of nucleic acid sequences that provides for transcription of an operably linked nucleic acid sequence. As used herein, the term “operably linked” refers to a juxtaposition of the components described, wherein the components are in a relationship that permits them to function in their intended manner. In general, operably linked components are in contiguous relation.

Expression constructs of the invention will also generally include regulatory elements that are functional in the intended host cell in which the expression construct is to be expressed. Thus, a person of ordinary skill in the art can select regulatory elements for use in, for example, bacterial host cells, yeast host cells, mammalian host cells, and human host cells. Regulatory elements include promoters, transcription termination sequences, translation termination sequences, enhancers, and polyadenylation elements.

An expression construct of the invention can comprise a promoter sequence operably linked to a polynucleotide sequence encoding a peptide of the invention. Promoters can be incorporated into a polynucleotide using standard techniques known in the art. Multiple copies of promoters or multiple promoters can be used in an expression construct of the invention. In a preferred embodiment, a promoter can be positioned about the same distance from the transcription start site as it is from the transcription start site in its natural genetic environment. Some variation in this distance is permitted without substantial decrease in promoter activity. A transcription start site is typically included in the expression construct.

Agents that Promote NK Cell Activity

In certain embodiments, agents that promote NK cell activity include, but are not limited to, agents that promote expansion and maturation of NK cells, and agents that enhance cytotoxicity of NK cells. Various agents that enhance NK cell activity are known in the art. In certain embodiments, agents that enhance NK cell activity include, but are not limited to, IL-15, IL-12, pectin, interferons (such as IFN-gamma), IL-2, and IL-21.

Interleukin-12 (IL-12) is a cytokine that promotes and stimulates activity of NK cells and T lymphocytes, and has antitumor activity (U.S. Patent Application Publication No. 2012/0251441).

Interleukin 15 (IL-15) is a cytokine that stimulates NK cells [Fehniger T A, Caligiuri M A. Blood 97(1):14-32 (2001)]. It has become apparent that IL-15 presented through cell-to-cell contact has a higher NK stimulating activity than soluble IL-15 [Dubois S, et al., Immunity 17(5):537-547 (2002); Kobayashi H, et al., Blood (2004) PMID: 15367431; Koka R, et al., J Immunol 173(6):3594-3598 (2004); Burkett P R, et al., J Exp Med 200(7):825-834 (2004)]. (U.S. Patent Application Publication No. 2012/0015434).

Pectin is generally a natural macromolecular polysaccharide polymer, widely distributed in plant cell walls (Hyunjo Kim, et al. International Journal of Pharmaceutics, 1998, 161:149-159). Pectin can improve the immune function of hosts by enhancing the mononuclear phagocyte system, activating macrophages, T cells, B cells, NK cells and complement system; promoting cytokine secretion; enhancing immunity of erythrocytes; and exerting direct anticancer effects by changing the growth characteristics of solid cancer cell membrane, affecting signal transmission path in solid cancer cells and anti-free radical effect, inducing differentiation and apoptosis, inhibiting synthesis of nucleic acid and protein of the solid cancer cells, affecting ultrastructure of the solid cancer cells, affecting oncogenes and antimutation effect, and inhibiting vascularization of solid cancer (Chinese Journal of Information on Traditional Chinese Medicine, 1999, 5:64) (U.S. Patent Application Publication No. 2012/0244193).

Interleukin 21 (IL-21) promotes the expansion and maturation of NK cells. (U.S. Patent Application Publication No. 2011/0268766).

Interferons (such as, IFN-gamma) and IL-2 have been shown to promote NK cell activity.

### Therapeutic Compositions and Routes of Administration

The subject invention further provides for therapeutic or pharmaceutical compositions.

In an embodiment, the composition comprises a therapeutically effective amount of a protein, nucleic acid molecule, and/or compound of the subject invention and, optionally, a pharmaceutically acceptable carrier.

The subject invention contemplates therapeutic compositions useful for practicing the therapeutic methods described herein. The therapeutic composition can be any form of pharmaceutical format, including injectable formulations such as liquid and lyophilized injections.

Suitable non-toxic pharmaceutically acceptable carriers for use with the agent will be apparent to those skilled in the art of pharmaceutical formulation. See, for example, Remington's Pharmaceutical Sciences, seventeenth edition, ed. Alfonso R. Gennaro, Mack Publishing Company, Easton, Pa. (1985). Suitable carriers include ethanol, dimethyl sulfoxide, glycerol, silica, alumina, starch, sorbitol, inosital, xylitol, D-xylose, manniol, powdered cellulose, microcrystalline cellulose, talc, colloidal silicon dioxide, calcium carbonate, magnesium cabonate, calcium phosphate, calcium aluminium silicate, aluminium hydroxide, sodium starch phosphate, lecithin, and equivalent carriers and diluents. Saline solutions and aqueous dextrose and glycerol solutions can also be employed as liquid carriers, particularly for injectable solutions.

Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. The therapeutic composition, if desired, can also contain minor amounts of wetting or emulsifying agents, or pH buffering agents.

The amount of active ingredient that may be combined with the carrier materials to produce a single dosage form will vary, depending on the type of the condition and the subject to be treated. In general, a therapeutic composition contains from about 5% to about 95% active ingredient (w/w). More specifically, a therapeutic composition contains from about 20% (w/w) to about 80%, or about 30% to about 70%, active ingredient (w/w).

The preparation of a pharmacological composition that contains active ingredients dissolved or dispersed therein is well understood in the art and need not be limited based on formulation. Typically such compositions are prepared as injectables either as liquid solutions or suspensions; however, solid forms suitable for solution, or suspensions, in liquid prior to use also can be prepared. The preparation also can be emulsified.

The therapeutic composition of the subject invention can include pharmaceutically acceptable salts of the components therein. Pharmaceutically acceptable salts include the acid addition salts (formed with the free amino groups of a polypeptide) that are formed with inorganic acids such as, for example, hydrochloric or phosphoric acids, or such organic acids as acetic, tartaric, mandelic and the like. Salts formed with the free carboxyl groups also can be derived from inorganic bases such as, for example, sodium, potassium, ammonium, calcium or ferric hydroxides, and such organic bases as isopropylamine, trimethylamine, 2-ethylamino ethanol, histidine, procaine and the like.

As used herein, the terms “pharmaceutically acceptable”, “physiologically tolerable” and grammatical variations thereof, as they refer to compositions, carriers, diluents and reagents, are used interchangeably and represent that the materials are capable of administration to or upon a mammal.

The invention also provides a pharmaceutical pack or kit comprising one or more containers filled with one or more of the ingredients, e.g., compound, carrier suitable for administration.

The compositions of the subject invention can be administered to the subject being treated by standard routes, including oral, inhalation, or parenteral administration including intravenous, subcutaneous, topical, transdermal, intradermal, transmucosal, intraperitoneal, intramuscular, intracapsular, intraorbital, intracardiac, transtracheal, subcutaneous, subcuticular, intraarticular, subcapsular, subarachnoid, intraspinal, epidural and intrasternal injection, infusion, and electroporation, as well as co-administration as a component of any medical device or object to be inserted (temporarily or permanently) into a subject.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi.

Sterile injectable solutions are prepared by incorporating the active ingredients in the required amount in the appropriate solvent followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum drying and freeze-drying techniques, which yield a powder of the active ingredient, plus any additional desired ingredient from a previously sterile-filtered solution thereof.

### Materials and Methods

### Cell Culture and Assays

Human HCC cell lines, Hep3B and HepG2, were purchased from American Type Culture Collection (Manassas, Va.) and were cultured; while Huh7 was purchased from Health Science Research Resources Bank (Osaka, Japan) and maintained in Dulbecco's modified Eagle medium (DMEM) supplemented with 10% heat-inactivated fetal bovine serum (Invitrogen, Carlsbad, Calif.).

Stable transfectant for GEP over-expression was established by transfecting GEP full-length eDNA into HepG2, a cell line with low endogenous GEP level; while GEP suppression was performed by transfecting GEP shRNA into Hep3B, a cell line with high endogenous GEP level. Both GEP over-expression and suppression transfectants were maintained in 10% AMEM with 0.4 mg/ml G418.

GEP blockage in Hep3B was performed by incubating the cells with or without 50 μg/ml anti-GEP monoclonal antibody A23 or mouse IgG isotype control antibody (Sigma-Aldrich, St. Louis, Mo.) for 24 h. GEP stimulation in HepG2 cells was performed by incubating the cells with or without 0.8 μg/ml recombinant GEP for 24 h.

### Clinical Specimens

The study protocol was approved by the Institutional Review Board of the University of Hong Kong/Hospital Authority Hong Kong West Cluster (HKU/HA HKW IRB). Five patients who underwent curative partial hepatectomy or liver transplantation for HCC at Queen Mary Hospital, Hong Kong, were recruited with written informed consent to the study. The patients had been diagnosed with primary HCC and confirmed by pathological examinations. Ethics approval for the use of archived fetal tissues identified from a computer database has been obtained from the Institutional Review Board of the University of Hong Kong/Hospital Authority Hong Kong West Cluster (HKU/HA HKW IRB). Tumor and adjacent non-tumor tissue pair from HCC patients were collected and examined for the expression of GEP, MICA and HLA-E by real-time quantitative reverse-transcription polymerase chain reaction (RT-PCR), immunohistochemistry and flow cytometry. Sera of HCC patients and healthy donors were collected for measurement of soluble MICA (sMICA) and GEP levels.

### Immunofluorescence Staining and Flow Cytometric Analysis

For HCC cell surface expression of ligands for NK cell immunoreceptors, cells were stained with APC-conjugated mouse anti-human MICA, HLA-A/B/C, HLA-G, PE-conjugated mouse anti-human HLA-E antibodies or equal amount of corresponding isotype control (BD Biosciences). For co-expression with GEP, cells were stained with the above surface markers, followed by permeabilization with ice-cold 0.1% saponin and then incubated with FITC-conjugated mouse anti-human GEP antibody (described previously) or equal amount of FITC-conjugated mouse IgG isotype antibody (Sigma). For NKG2D surface expression on NK cells, cells were stained with APC-conjugated mouse anti-NKG2D antibody or equal amount of corresponding isotype control (BD Biosciences). Results were expressed as percentage of cells positive for the above markers and/or mean fluorescence intensity (MFI) of the markers, after subtracting the non-specific background signal (isotype controls).

### Preparation of Human Effector Cells

Human peripheral blood mononuclear cells (PBMC) were freshly isolated from the huffy coats of healthy donors by density gradient centrifugation using Ficoll-Paque Plus. NK cells were isolated from PBMC using magnetic cell sorting by positive selection with anti-CD56 microbeads, while the unlabeled flow-through were the NK cell-depleted PBMCs, according to the manufacturer's instructions (Miltenyi Biotech). The resulting PBMCs, NK cells, and NK cell-depleted PBMCs were washed with RPMI 1640 with 10% FBS and used as human effector cells in the cell cytotoxicity assay.

### Cell Cytotoxicity Assay

Cell cytotoxicity was determined by dual-color flow cytometry using the HCC cell lines Hep3B or HepG2 as target cells, while PBMC, NK cells, or NK cell-depleted PBMCs were used as effector cells. Briefly, HCC cells (1×10⁶ cells/ml) were labeled with the green fluorescence cytoplasmic dye 5- and (6)-carboxyfluorescein diacetate, succinimidylester (CSFE) at 0.1 μM for 15 min at 37° C. in dark. After washing, CFSE-labeled HCC cells were then co-cultured with or without effector cells at indicated effector to target cells ratio (E/T ratio) in RPMI supplemented with 10% FBS for additional 5 hr at 37° C. in 5% C02. Cells were then collected and stained with 10 μg/ml propidium iodide (PI) for 15 min at room temperature in dark. Cells were subject to dual-color flow cytometric analysis, with lysed target cells recognized as CFSE⁺PI⁺, while viable target cells as CFSE⁺PI⁻. Cell cytotoxicity level was expressed as percentage of lysed cells within the CFSE⁺ target cells (Hep3B or HepG2), and calculated as follows:

Percentage of CFSE⁺PI⁺ cells I (Percentage of CFSE⁺PI⁺ cells plus percentage of CFSE⁺PI⁻ cells)×100.

For blocking experiments, neutralization of MICA was performed by incubating the CFSE-labeled HCC cells with or without anti-MICA neutralizing antibody or mouse IgG2a isotype control antibodies (2 ug/ml, Biolegend) and co-cultured with effector cells.

For antibody-dependent cytotoxicity (ADCC), CFSE-labeled HCC cells were stained with or without indicated concentrations of anti-GEP antibody A23, or mouse IgG isotype antibody for 30 min at 4° C. Labeled target cells were then co-cultured with effector cells at indicated E/T ratio.

### Orthotopic Mouse Model

The establishment of an orthotopic mouse model and drug treatments was described (Wong N C et al. Mol Cancer Therapeutics MS# MCT-14-0012 in revision). Briefly, Hep3B cells transfected with firefly luciferase activity, were injected subcutaneously into the right flank of 4-5 weeks old male BALB/c nu/nu (nude) mice and were observed daily for signs of tumor development. Once the subcutaneous tumor reached 1-1.5 cm in diameter, it was removed, cut into 1-2 mm cubes and implanted into the left liver lobe of another group of 4-5 weeks old nude mice under anesthesia. The size of tumor was monitored by the in vivo imaging system, IVIS100 (Caliper Life Sciences, Hopkinton, Mass., USA) with intra-peritoneal (i.p.) injection of 150 mg/kg of D-Luciferin (Gold Biotechnology, St Louis, Mo., USA) under anesthesia. The tumor take rate was confirmed and experiments were started with ROI greater than 1×10⁶ p/s/cm2/sr. The bioluminescent signal intensity emitted from the intrahepatic tumor was quantified weekly with Living Image Software (Caliper Life Sciences). Mice bearing intrahepatic Hep3B tumors were randomized and received i.p. injection of saline (control) (n=3) or 10 mg/kg anti-GEP antibody twice weekly (n=3). Mice were sacrificed after six weeks of treatment. Tumors were dissected and embedded with paraffin for further analysis.

### Enzyme-Linked Immunosorbent Assay (ELISA) to Determine GEP and MICA Levels

Soluble MICA levels in culture supernatants and human serum samples were determined by DuoSet MICA ELISA kit (R&D Systems, Minneapolis, Minn.). GEP levels in human serum samples were detected by human progranulin ELISA kit (Adipogen Inc., Seoul, Korea).

### Immunohistochemical Staining

Immunohistochemistry was performed with the Dako Envision Plus System (Dako, Carpinteria, Calif.) following the manufacturer's instruction with modifications. Briefly, antigen retrieval was performed by microwave with sections immersed in citrate buffer. Followed by endogenous peroxidase blocking, tissues were stained with mouse anti-human GEP antibody (described previously), goat anti-human MICA (R&D Systems) and mouse anti-human HLA-E (Abcam, Cambridge, Mass.). The signal was detected by horseradish peroxidase-conjugated secondary antibody and color was developed with diaminobenzidine as the chromogen. The tissue sections were then counterstained with hematoxylin.

### Real-Time Quantitative Reverse-Transcription Polymerase Chain Reaction

Real-time quantitative RT-PCR was performed as described previously (Cheung S T et al., Clin Cancer Res. 2004). Quantification was performed with the ABI Prism 7700 sequence detection system (Applied Biosystems, Foster City, Calif.). Primers and probe for GEP have previously been described. Primers and probe were ready-made reagents (Pre-Developed TaqMan Assay Reagents; Applied Biosystems) for MICA (catalog no./assay ID: Hs00792195_m1), HLA-E (catalog no./assay ID: Hs03045171_m1), and control 18 s. In brief, there were 40 amplification cycles. The sample would be regarded as absent GEP, MICA or HLA-E expression when there was no detectable signal of GEP, MICA or HLA-E up to 40 cycles. The relative amount of GEP, MICA and HLA-E, which had been normalized with control 18 s for RNA amount variation and calibrator for plate-to-plate variation, was presented as the relative fold change.

### Statistical Analyses

All analyses were performed using the statistical software GraphPad Prism Version 3.00 for Windows (GraphPad Software, CA, USA) or SPSS version 16.0 for Windows (SPSS Inc, Chicago, Ill.). All in vitro data were expressed as mean values±standard deviation (SD) from at least three independent experiments. Continuous variables were assessed by Spearman correlation and compared between groups by ANOVA (clinical samples) or Student t test (in vitro models). GEP, MICA and HLA-E transcript levels were continuous variables, and the data were modeled as categorical variables in Kaplan-Meier and Cox regression analyses. The Youden index (i.e., sensitivity+specificity−1) was used to determine the optimal cutoff point for the prediction of 3-year recurrence-free survival maximizing the sensitivity and the specificity of the prediction. The association of GEP, MICA, HLA-E and tumor stage (American Joint Committee on Cancer tumor staging system version 7) with recurrence-free survival was examined by univariable and multivariable Cox proportional hazards regression with a forward stepwise selection procedure. P value <0.05 was considered statistical significant.

### EXAMPLES

Following are examples that illustrate procedures for practicing the invention. These examples should not be construed as limiting.

### Example 1

### GEP Expression of HCC Cells Modulates Anti-Tumor Cytotoxic Activity of Immune Cells

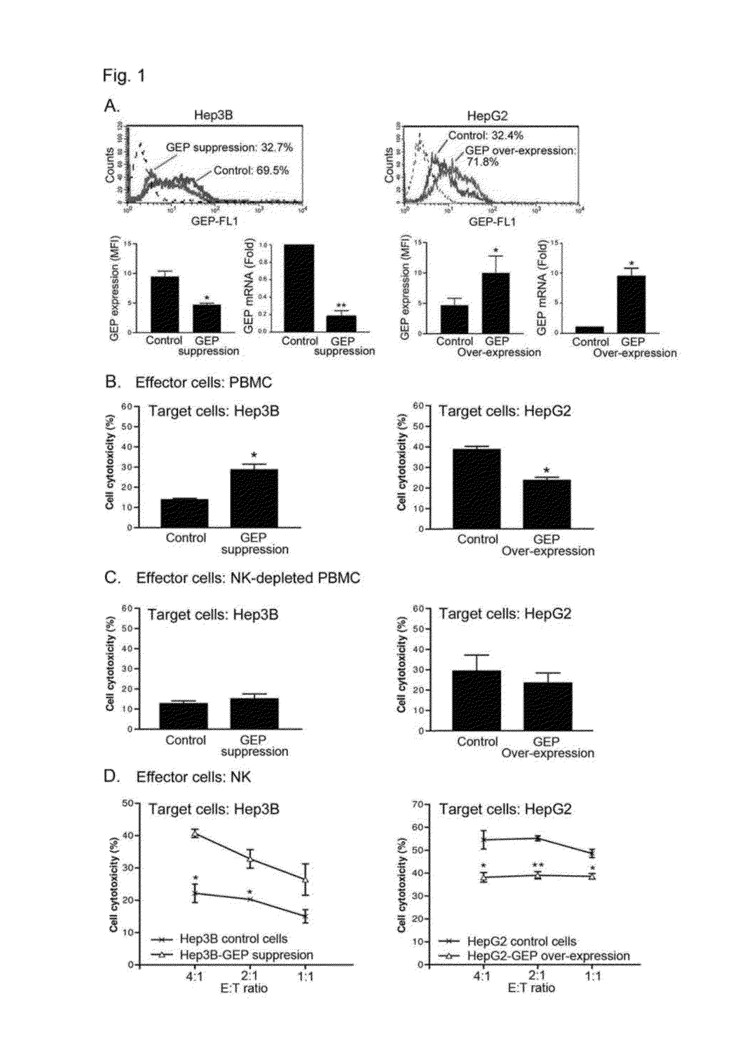

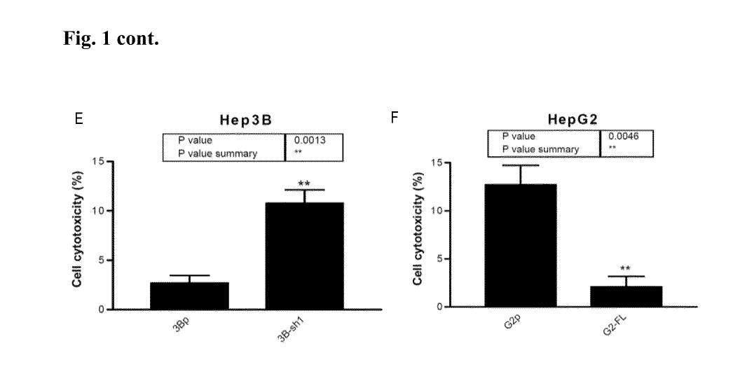

It is postulated that only a restricted minority of tumorigenic malignant cells may possess the characteristics needed to modulate tumor-directed immune activation. To examine the effect of GEP on anti-tumor immunity, a stable transfectant of GEP suppression was established using shRNA in Hep3B, a HCC cell line with high endogenous GEP; a stable transfectant of GEP over-expression was established using GEP full-length eDNA in HepG2, a HCC cell line with low endogenous GEP level. Results from qPCR and flow cytometry showed that GEP mRNA and protein levels were significantly down-regulated in Hep3B cells, and up-regulated in HepG2 cells (FIG. 1A).

Cytotoxic activity of human PBMC against the HCC cells and their GEP transfectants was examined by dual-color flow cytometry. Cytotoxic activity of human PBMC against GEP-suppressed Hep3B cells was significantly higher than control Hep3B cells. On the contrary, cytotoxic activity of PBMC against HepG2 cells was significantly reduced when GEP was over-expressed.

The results show that GEP expression is crucial for rendering HCC cells resistant to anti-tumor cytotoxic activity of PBMC (FIG. 1B).

PBMC encompasses a variety of immune cells including T and B lymphocytes, monocytes, natural killer (NK) cells, and dendritic cells. The anti-tumor activity of NK cells in liver has been well documented. Clinical studies also suggest that NK cells contribute to innate defenses against primary liver tumors and liver metastases in patients.

To evaluate whether NK cells are involved in the GEP-modulated immunoevasion, CD56⁺ NK cells were depleted from human PBMC by magnetic sorting. Upon NK cell depletion, the increased cytotoxic activity against GEP-suppressed Hep3B cells was abolished; while the reduction of cytotoxic activity against GEP over-expressed HepG2 was restored (FIG. 1C).

To further confirm the role of NK cells, HCC cells and their GEP transfectants were co-cultured with NK cells at different E:T ratio. Cytotoxic activity of NK cells against GEP-suppressed Hep3B cells was significantly higher than that against control Hep3B cells; while significant reduction in NK cytotoxic activity was demonstrated in GEP over-expressed HepG2 cells when compared with control HepG2 cells. Besides, cytotoxicity against HCC cells increased as the number of NK cells increased (FIG. 1D). The results show that the GEP-regulated anti-tumor cytotoxicity was dependent on NK cells.

Since the NK cells of the previously described studies herein were isolated from lymphocytes of healthy individuals, it was necessary to determine if the same effect is observed when the NK cells are isolated from liver cancer patients. NK cells isolated from liver cancer patients showed enhanced cytotoxicity in liver cancer cells with GEP suppression (Hep3B cells transfected with GEP shRNA, 3B-sh1) (FIG. 1E) and showed decreased cytotoxicity in liver cancer cells with GEP over-expression (HepG2 transfected with GEP full-length, G2FL) (FIG. 1F). As such, activation of NK cells in liver cancer patients, by GEP antibody or other inhibitors, could result in control of tumor growth without transfusion of NK cells from healthy counterparts.

### Example 2

### GEP Differentially Regulates the Expression of MICA and HLA-E of HCC Cells

NK activity is regulated through integrated signaling from a panel of stimulatory and inhibitory receptors that interact with their ligands expressed on tumor cells (Lanier, L L. Annu Rev Immunol. 2005). To elucidate the mechanism for GEP-regulated NK cytotoxicity, the effect of GEP on HCC cell surface expression of ligands for both stimulatory and inhibitory receptors of NK cells was determined. MICA and HLA-A/B/C are ligands for stimulatory immunoreceptor NKG2D, and KIR2DL1/3DL1; while HLA-E and HLA-G are ligands for inhibiting immunoreceptor NKG2A and KIR2DL4, respectively. The surface expression of MICA, HLA-A/B/C, HLA-E, and HLA-G on Hep3B and HepG2 cells was measured, and their GEP transfectants by flow cytometry.

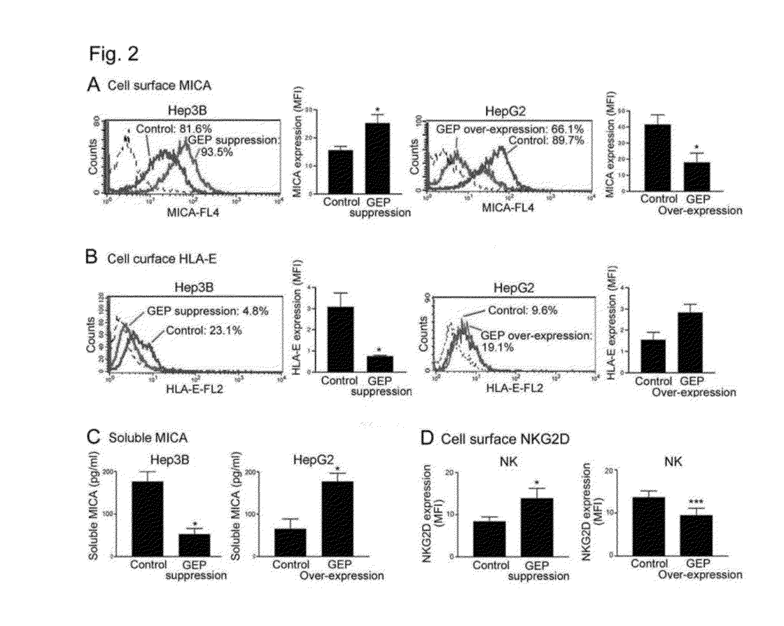

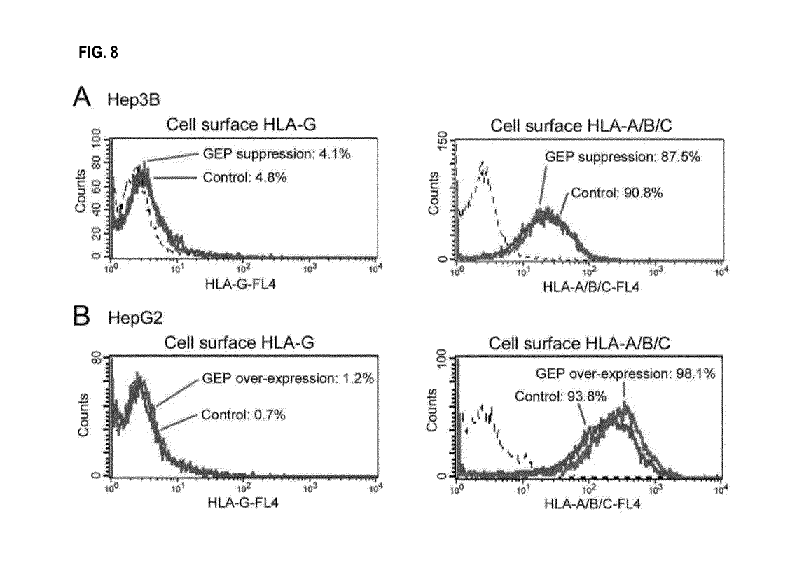

Upon GEP suppression, expression of surface MICA significantly increased (FIG. 2A), while surface HLA-E expression significantly decreased when compared with control Hep3B cells (FIG. 2B). When GEP was over-expressed in HepG2, expression of surface MICA significantly decreased (FIG. 2A), while surface HLA-E level significantly increased when compared with control cells (FIG. 2B). However, no prominent change was observed for HLA-A/B/C and HLA-G expression upon GEP modulation in both cell lines (FIG. 8). To further study the effect of GEP on MICA expression, the soluble MICA in the culture supernatants of HCC cells and their GEP transfectants was measured. GEP suppression in Hep3B decreased; while GEP over-expression in HepG2 increased soluble MICA release significantly (FIG. 2C), suggesting that GEP regulated surface MICA levels at least partially by controlling its shedding from HCC cells.

Since soluble MICA was shown to suppress NKG2D expression on NK cells (Nausch, N and Cerwenka, A. Oncogene. 2008), the effect of GEP modulation on the expression of NKG2D on NK cell surface upon co-culture with HCC cells was examined. Upon co-culture with GEP-suppressed Hep3B cells, surface NKG2D levels on NK cells significantly increased when compared with those co-cultured with Hep3B control cells. When NK cells were co-cultured with HepG2 cells with GEP over-expression, their surface NKG2D levels significantly decreased when compared with those co-cultured with HepG2 control cells (FIG. 2D).

### Example 3

### GEP Blockage by Anti-GEP Antibody A23 Modulates the Expression of MICA and HLA-E of HCC Cells

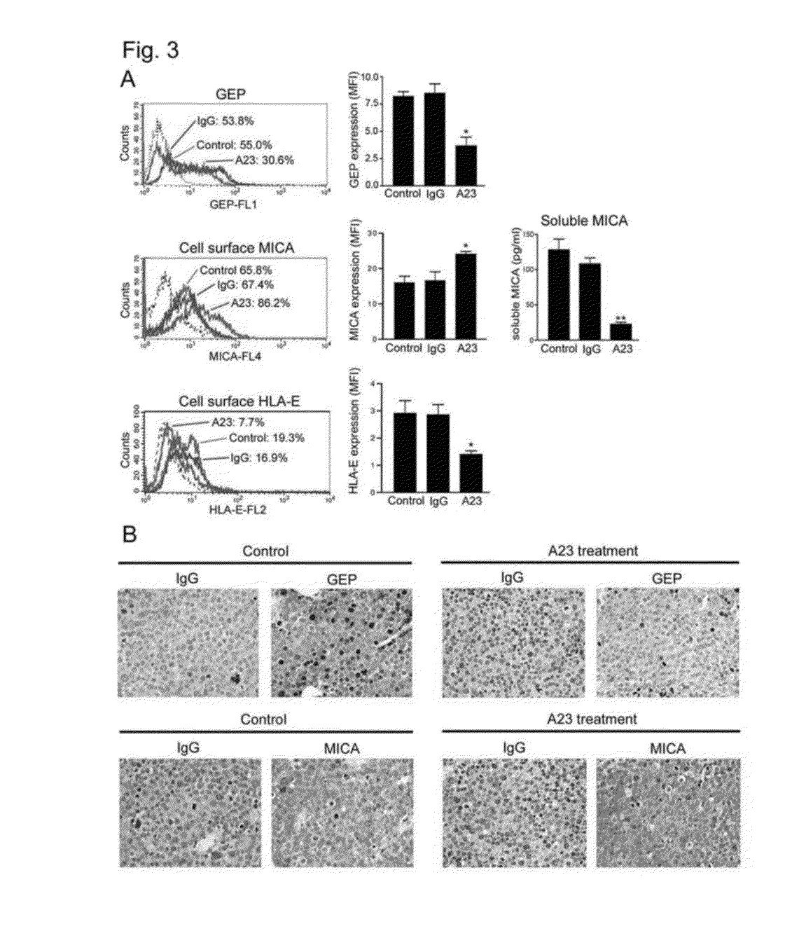

To further validate the regulatory role of GEP on the expression of MICA and HLA-E, GEP blockage by anti-GEP monoclonal antibody A23 was performed in Hep3B cells. A23, but not mouse IgG, significantly suppressed GEP expression of Hep3B cells (FIG. 3A), demonstrating the suppressing effect was specific. A23 was found to significantly increase the expression of surface MICA, while decrease soluble MICA release and surface HLA-E expression of Hep3B when compared with control and isotype control treatment (FIG. 3B).

To confirm the regulatory role of anti-GEP antibody A23 in vivo, orthotopic tumors were generated of Hep3B cells in nude mice. Tumor bearing mice were subject to saline (control) or 10 mg/kg anti-GEP antibody twice weekly for six weeks. No signs of disability and behavior abnormalities were observed during the treatment period. GEP antibody treatment was found to significantly suppress tumor growth when compared with saline control in terms of tumor volumes and tumor signals (Wong, N C et al. submitted manuscript). Protein expression of GEP, MICA and HLA-E in the tumors was detected by immunohistochemical staining A23 was found to markedly reduce the protein expression of GEP, but increase that of MICA significantly when compared with saline control (FIG. 3B). However, HLA-E protein could not be detected in the xenograft tumors (data not shown), which was probably due to the low constitutive expression level of HLA-E protein in Hep3B cells. The results indicated that A23 could reduce tumor growth in vivo, which might be at least partially due to the suppression of GEP and up-regulation of MICA levels of HCC cells, thereby facilitating NK cytotoxicity against the tumors.

### Example 4

### GEP Blockage by Anti-GEP Antibody A23 Enhances NK Cell Cytotoxicity Against HCC Cells Via MICA

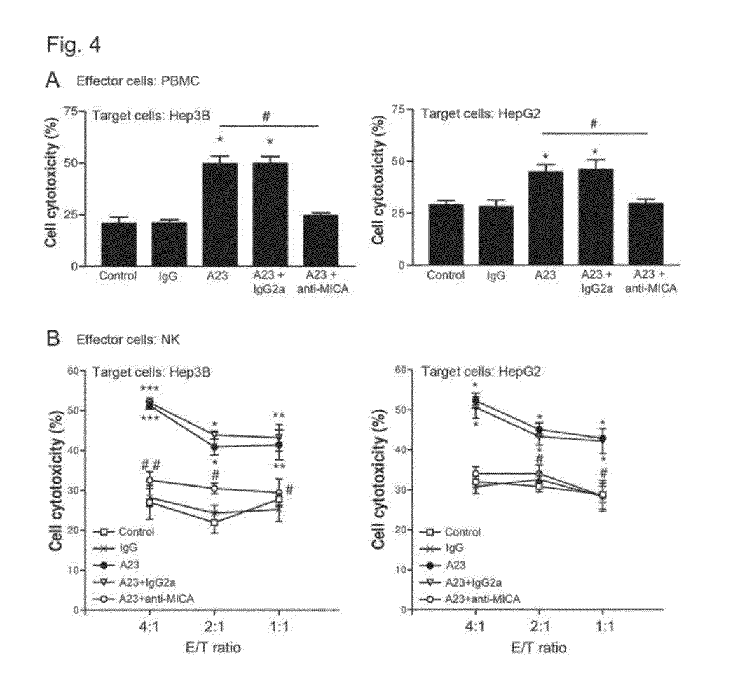

Confirming the role of GEP in tumor immune evasion, the therapeutic potential of anti-GEP monoclonal antibody A23 in anti-tumor immunity in HCC was studied. HCC cells were treated with A23 or isotype control antibody for 24 h prior to co-culture with PBMC to assess the effect of A23 on anti-tumor cytotoxic activity against HCC cells. Results showed that cytotoxic activities of PBMC against A23-treated Hep3B and HepG2 cells were significantly higher than those of control and isotype control antibody-treated cells (FIG. 4A). Note that the increase in HepG2 cells that were sensitized to PMBC cytotoxicity upon A23 treatment was smaller than those observed in Hep3B. This is probably because HepG2 cells constitutively express lower level of endogenous GEP than Hep3B cells, the magnitude of GEP reduction by A23 in HepG2 is smaller than that in Hep3B, resulting to the smaller magnitude of A23-induced effect observed in HepG2.

To examine the role of MICA in A23-induced PBMC cytotoxicity against HCC cells, blocking antibody of MICA was used. HCC cells were treated with A23 for 24 h and then incubated with anti-MICA neutralizing antibody or isotype control (mouse IgG2a) and co-cultured with PBMC. Addition of anti-MICA blocking antibody, but not mouse IgG2a isotype control, could markedly abrogate the A23-induced cytotoxicity against Hep3B and HepG2 cells. The results suggested that the A23-induced cytotoxic activity against HCC cells was dependent on MICA. (FIG. 4A)

Since the MICA-NKG2D signal is important for NK cell activation, the effect of MICA neutralization on A23-induced NK sensitivity of HCC cells was studied. Results showed that blocking MICA could significantly abolish the A23-induced NK sensitivity of HCC cells at different E:T ratios, therefore confirming that A23-induced NK cytotoxicity against HCC cells was dependent on MICA (FIG. 4B).

### Example 5

### Antibody-Dependent Cellular Cytotoxicity Mediated by Anti-GEP Monoclonal Antibody A23

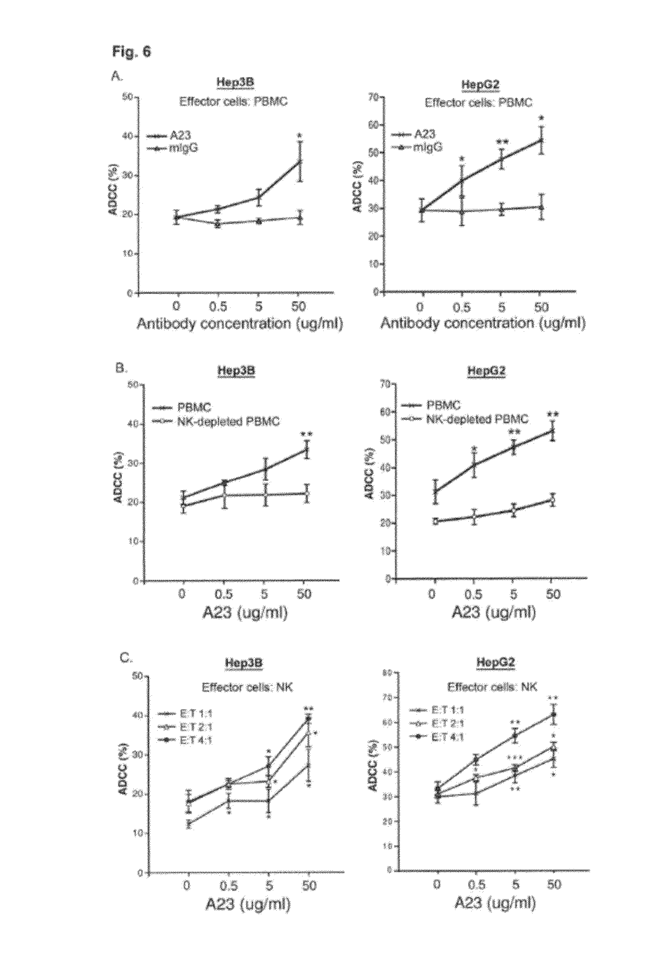

To further characterize the immunomodulatory mechanism of anti-GEP monoclonal antibody A23, antibody-dependent cellular cytotoxicity (ADCC) of A23 was assessed. ADCC occurs when antibodies bind to antigen on tumor cells and the antibody Fc domains engage Fc receptors on the surface of immune effector cells. Hep3B or HepG2 cells were stained with or without anti-GEP antibody A23 or mouse isotype control antibody for 30 min and the antibody-labeled cancer cells were then co-cultured with PBMC. A23, but not isotype control antibody, significantly induced ADCC of human PBMC against both Hep3B and HepG2 cells in a dose-dependent manner (FIG. 6A). The results show that the anti-tumor activity of A23 was at least partly attributable to ADCC.

In addition, this Example further elucidates which effector cells are important for the ADCC activity of A23 in human. NK cells were depleted from PBMC and the A23-mediated ADCC in both cell lines was markedly abolished upon depletion of NK cells from PBMC (FIG. 6B). A23-labeled cancer cells were then co-cultured with NK cells and the A23-mediated ADCC against the HCC cells increased along the E/T ratio (FIG. 6C). Therefore, it was confirmed that NK cells also play an important role as effector cells for the A23-induced ADCC in human.

### Example 6

### Expression Patterns of GEP, MICA and HLA-E in HCC Clinical Specimens

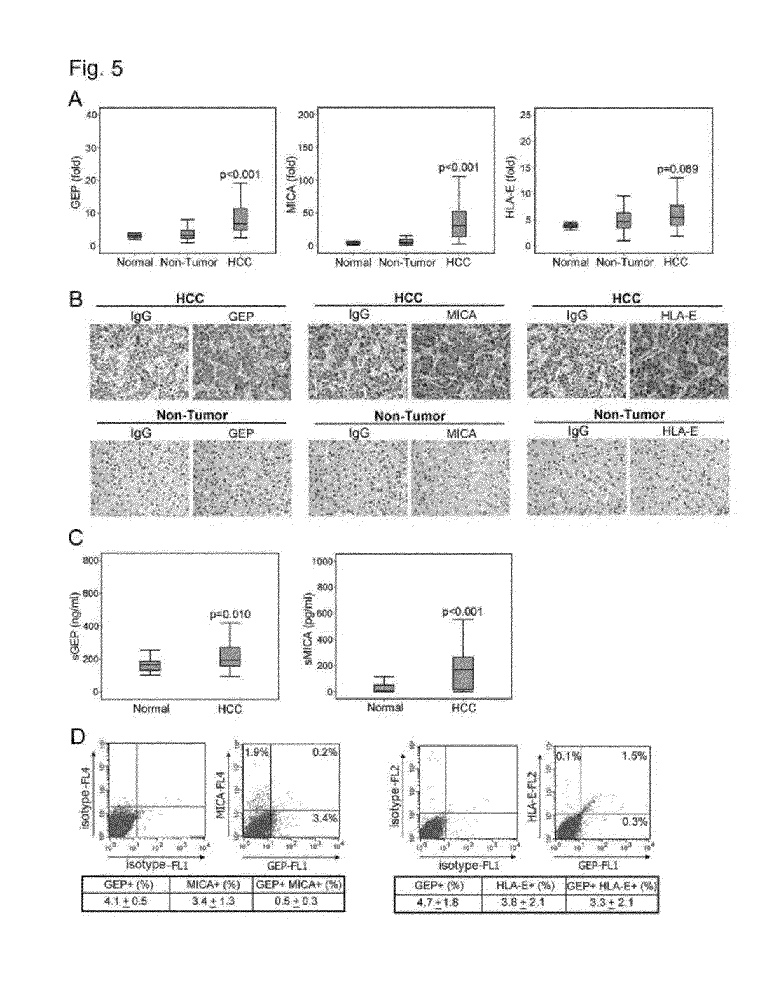

GEP and MICA transcript levels were found to be significantly elevated in HCC when compared with paralleled tumor adjacent non-tumor liver tissues and normal livers from healthy individuals (both p<0.001). HLA-E transcript level was also elevated in HCC when compared with non-tumor liver tissues and normal livers, though statistical significance was not reached (p=0.089) (FIG. 5A). By immunohistochemical staining, it was revealed that total protein expression levels of GEP, MICA and HLA-E were stronger in HCC when compared with their adjacent non-tumor liver tissues (FIG. 5B). Besides, serum GEP and MICA levels were found to significantly higher in HCC patients when compared with healthy individuals (p=0.010 and p<0.001, respectively) (FIG. 5C).

It is shown that GEP conferred HCC cells resistance to NK cytotoxicity by differentially regulated surface MICA and HLA-E expression in HCC cell lines. To confirm the immunoevasion ability of GEP-expressing cells in clinical settings, cell surface MICA and HLA-E were co-stained with GEP in HCC clinical specimens by immunofluorescence staining and flow cytometry. Results showed that expressions of surface MICA and GEP were mutually exclusive (n=3) (FIG. 5D). However, surface HLA-E were found to co-express with GEP in the HCC clinical specimens (n=3) (FIG. 5D). The result therefore echoes that of cell line model that GEP down-regulated MICA, but up-regulated HLA-E on the surface of HCC cells, rendering the cells less susceptible to NK cytotoxicity. Note that in primary HCC fresh tissues, GEP+ cells only contributed to less than 5% of the whole tumor bulk, which was comparable to the other hepatic CSC markers such as CD133 (Ma S et al. Cell stem cell. 2010) and CD90 (Yang Z F, et al. Cancer Cell. 2008). Such small proportion was actually in accordance with the definition of CSCs, that only a minority of cells in the tumor bulk was able to self-renew and regenerate the tumors (Reya, T et al. Nature. 2001).

### Example 7

### Association of Serum GEP and MICA Levels with Clinico-Pathological Parameters

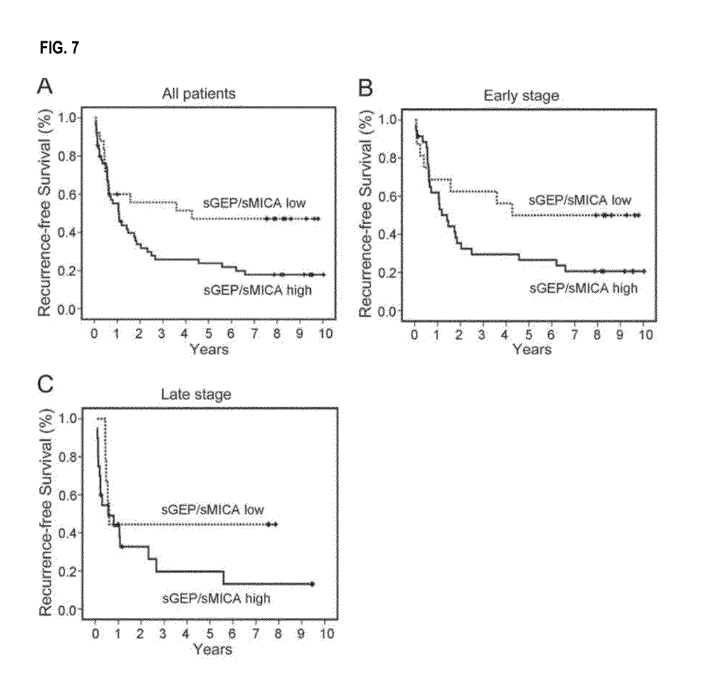

To investigate the potential of GEP and MICA as predictive or prognosis markers for HCC, the association of serum GEP and MICA levels with clinico-pathological parameters in HCC patients (n=80) was determined. Here, it was found that serum GEP levels significantly correlated with tumor size (HCC n=80, Spearman's p correlation coefficient=0.569, p<0.001) (Table 1). Previous study has suggested serum MICA level as a potential prognostic marker for HBV-induced HCC (Kumar, V et al. PLoS One. 2012). However, no significant association was found between serum MICA level and HBV infection in the patient cohort. Kaplan-Meier plot was used to examine patient outcome in association with serum GEP and MICA levels. Patients were segregated into low expression group (low in both serum GEP and MICA) and high expression group (either one or both high in serum GEP and MICA) with the Youden index maximized to determine optimal cutoff value. There were 25 patients in the low expression group (median recurrence-free survival, 51.2 months) and 55 patients in the high expression group (median recurrence-free survival, 12.8 months). Patients with high serum GEP and/or MICA levels were found to have poor recurrence-free survival (log-rank test, p=0.042) (FIG. 7A). When the patients were segregated into early (FIG. 7B) and advanced (FIG. 7C) tumor stages, patients with high serum GEP and/or MICA levels were also found to have poor recurrence-free survival.

TABLE 1
Clinico-pathological features of HCC
in relation to sGEP and sMICA levels.

| Clinico-pathological | sGEP level | | | sMICA level | | |
|---|---|---|---|---|---|---|
| variables | Low | High | P value | Low | High | P value |
| Venous infiltration | | | | | | |
| Absence | 23 | 16 | 0.179 | 20 | 19 | 1.000 |
| Presence | 17 | 24 | | 20 | 21 | |
| Tumor size | | | | | | |
| Small (≦5 cm) | 18 | 5 | 0.003** | 10 | 13 | 0.622 |
| Large (>5 cm) | 22 | 35 | | 30 | 27 | |
| Tumor capsule | | | | | | |
| Absence | 11 | 11 | 0.712 | 10 | 12 | 0.067 |
| Presence | 4 | 7 | | 9 | 2 | |
| pTNM stage | | | | | | |
| Early stage (I-II) | 26 | 25 | 1.000 | 27 | 24 | 0.642 |
| Late stage (III-IV) | 14 | 15 | | 13 | 16 | |
| Gender | | | | | | |
| Male | 30 | 30 | 1.000 | 30 | 30 | 1.000 |
| Female | 10 | 10 | | 10 | 10 | |
| Age | | | | | | |
| Young (≦60) | 27 | 26 | 1.000 | 27 | 26 | 1.000 |
| Elderly (>60) | 13 | 14 | | 13 | 14 | |
| Serum AFP level | | | | | | |
| Low (≦20 ng/ml) | 14 | 16 | 0.818 | 13 | 17 | 0.489 |
| High (>20 ng/ml) | 26 | 24 | | 27 | 23 | |
| HBV association | | | | | | |
| Positive for HBsAg | 37 | 29 | 0.037 | 30 | 36 | 0.319 |
| Negative for HBsAg | 3 | 10 | | 9 | 4 | |

**P < 0.01
Abbreviations:
AFP, α-fetoprotein; HBV, hepatitis B virus; HBsAg, hepatitis B surface antigen

To examine the prediction power for recurrence-free survival, Cox regression analysis was used to compare the serum GEP/MICA data and tumor stage. By univariable Cox regression analysis, high expression level of serum GEP and/or MICA (hazard ratio [HR], 1.9; 95% confidence interval [CI], 1.0-3.5; p=0.045) and advanced tumor stage (hazard ratio [HR], 2.3; 95% confidence interval [CI], 1.5-3.5; p<0.001) were significantly associated with poor recurrence-free survival. By multivariable Cox regression analysis, only high expression level of sGEP and/or sMICA (HR, 1.9; 95% CI, 1.0-3.5; p=0.045), but not advance tumor stage (p=0.126), was found to be independent prognostic factors for recurrence-free survival (Table 2). The above findings showed that high sGEP and/or sMICA levels influenced the prognosis of HCC patients undergoing curative partial hepatectomy.

TABLE 2
Cox regression analyses for recurrence-free
survival on sGEP/MICA levels and tumor stage.

| | | Univariable analysis | | | Multivariable analysis | | |
|---|---|---|---|---|---|---|---|
| | | | | P | adjusted | | P |
| Variables \hat{ } | n | HR | (95% CI) | value | HR | (95% CI) | value |
| sGEP/ | | | | | | | |
| sMICA | | | | | | | |
| Low | 25 | 1 | | | 1 | | |
| High | 55 | 1.9 | (1.0-3.5) | 0.045 | 1.9 | (1.0-3.5) | 0.045 |
| Tumor | | | | | | | |
| stage | | | | | | | |
| Early | 51 | 1 | | | — | | |
| Late | 29 | 2.3 | (1.5-3.5) | <0.001 | — | — | — |

\hat{ } The sGEP/sMICA levels examined by ELISA were modeled as categorical variables in the analyses. The optimal cutoff value to segregate was determined by maximizing the Youden index.

### DISCUSSION

As a hallmark of tumor progression and recurrence, tumors have developed diverse mechanisms to evade the immune system (Hanahan, D and Weinberg, R A. Cell. 2011). Tumor cells have evolved to down-regulate their surface MICA by proteolytic shedding, therefore reducing their susceptibility of NKG2D-mediated NK cytotoxicity (Nausch, N and Cerwenka A. Oncogene. 2008). In this study, it was shown that GEP down-regulated MICA levels on HCC cell surface, therefore rendering GEP-expressing cells less susceptible to NK cytotoxicity. Besides, GEP over-expression promoted the production of soluble MICA (sMICA) from HCC cells. sMICA released from tumor cell surface can not only block the NKG2D-binding site for other NKG2D ligands, but also suppress NKG2D expression on NK cells (Nausch, N and Cerwenka A. Oncogene. 2008). Production of sMICA therefore represents one of the mechanisms in tumor immunoevasion (Bauer, S et al. Science. 1999; Salih, H R et al. J Immunol. 2002). Here, it was also shown that upon co-culture with HCC cells over-expressed with GEP, surface NKG2D on NK cells significantly decreased (FIG. 2D). Hence, GEP might confer HCC cells immunoevasion ability by promoting shedding of surface MICA and promote release of sMICA which perturbs NK cytotoxicity.

sMICA were frequently elevated in advanced HCC patients (Jinushi, M, et al. Journal of Hepatology. 2005; Kumar, V. et al. PLoS One. 2012; Li, J J et al. Chin J Cancer. 2012; Kohga, K et al. Cancer Sci. 2008; Kohga, K et al. Cancer Res. 2009), and such elevation was associated with down-regulated NKG2D expression and impaired NK activity in HCC (Jinushi, M et al. Journal of Hepatology. 2005). sMICA was reported as predictive and prognostic marker in HBV-induced HCC and advanced HCC (Kumar, V. et al. PLoS One. 2012; Li, J J et al. Chin J Cancer. 2012). To investigate the potential of sGEP and sMICA as predictive or prognosis markers for HCC, the association of sGEP and sMICA levels with clinicopathological parameters in HCC patients (n=80) was determined. Here, it was found that sGEP levels significantly correlated with tumor size. Besides, patients with high sGEP and sMICA levels have poor recurrence-free survival in early and advanced tumor staged patients. Further analysis by Cox regression confirmed that high sGEP and sMICA levels were independent prognostic factors for recurrence-free survival. Therefore, sGEP and sMICA levels can be used as potential prognostic markers for HCC patients.

Proteolytic cleavage and/or exosome secretion has been reported for shedding of MICA (Salih, H R et al. J Immunol. 2002; Groh, V et al. Nature. 2002). Matrix metalloproteinases (MMP) 9 and 14 were shown to mediate MICA shedding in various human cancers (Sun, D et al. Cell Biol Int. 2011; Liu, G et al. J Immunol. 2010). MMP9 is produced in a latent form, pro-MMP9, which requires activation to achieve catalytic activity. GEP was reported in ovarian cancer cell line model to regulate activation of MMP2 (Liu, Y et al. BMC Cancer. 2007), which in turn, was an activator of pro-MMP9 (Toth, M et al. Biochem Biophys Res Commun. 2003). Therefore, GEP might promote shedding of MICA from HCC cells by regulating MMP2 activation. Besides, it was previously found that transcript levels of GEP and MMP14 were significantly correlated in HCC tissues (unpublished data). However, further investigation is needed to clarify the mechanism underlying the cleavage of MICA from GEP-expressing HCC cells.

In addition to MICA, the unique long 16-binding protein (ULBP) family is also important ligand for NKG2D in human (Cosman, D et al. Immunity. 2001). Of which, ULBP1 was reported to express abundantly in HCC, and the loss of ULBP1 expression was significantly associated with early recurrence of HCC after hepatic resection (Kamimura, H et al. J Hepatol. 2012). However, there is no association between ULBP1 expression and overall survival of HCC patients. Although the loss of ULBP1 expression in poorly-differentiated HCC was shown to be induced by over-expression of proteasomes (Kamimura, H et al. J Hepatol. 2012), the mechanism involved is still unknown. The relationship between GEP and ULBP1 expression in HCC would be an intriguing area to be explored.

Up-regulation of HLA-E was suggested to represent a mechanism of tumor escape from NK cytotoxicity in ovarian cancer and melanoma (Malmberg, K J et al. J Clin Invest. 2002; Speiser, D E et al. J Exp Med. 1999). HLA-E mRNA was reported to be up-regulated in HCC tissues when compared with their adjacent non-tumor counterparts (Chen, A et al. Neoplasma. 2011). Besides, HLA-E polymorphism was associated with risk of hepatitis B or HCC (Zhang, J et al. Immunogenetics. 2012). However, the role of HLA-E in HCC pathogenesis and the cell populations over-expressing HLA-E have not been elucidated in HCC yet. Here, it is shown that GEP expression up-regulated HCC cells' surface expression of HLA-E. HLA-E might therefore play role in HCC pathogenesis by promoting immunoevasion ability of GEP-expressing cells.

All patents, patent applications, provisional applications, and publications referred to or cited herein are incorporated by reference in their entirety, including all figures and tables, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

### REFERENCES

1. Waldhauer I, Steinle A. NK cells and cancer immunosurveillance. Oncogene. 2008; 27:5932-43.
2. Lanier L L. NK cell recognition. Annu Rev Immunol. 2005; 23:225-74.
3. Jinushi M, Takehara T, Tatsumi T, Hiramatsu N, Sakamori R, Yamaguchi S, et al. Impairment of natural killer cell and dendritic cell functions by the soluble form of MHC class I related chain A in advanced human hepatocellular carcinomas. Journal of Hepatology. 2005; 43:1013-20.
4. Subleski J J, Hall V L, Back T C, Ortaldo J R, Wiltrout R H. Enhanced antitumor response by divergent modulation of natural killer and natural killer T cells in the liver. Cancer Res. 2006; 66:11005-12.
5. Nausch N, Cerwenka A. NKG2D ligands in tumor immunity. Oncogene. 2008; 27:5944-58.
6. Bateman A, Bennett HP. The granulin gene family: from cancer to dementia. Bioessays. 2009; 31:1245-54.
7. Cheung S T, Wong S Y, Leung K L, Chen X, So S, Ng I O, et al. Granulin-epithelin precursor overexpression promotes growth and invasion of hepatocellular carcinoma. Clin Cancer Res. 2004; 10:7629-36.
8. Cheung S T, Cheung P F, Cheng K C, Wong N C, Fan S T. GEP and ABCB5 Regulate Liver Cancer Chemoresistance. Gastroenterology. 2010; 140:344-55.
9. Cheung S T, Wong S Y, Lee Y T, Fan S T. GEP associates with wild-type p53 in hepatocellular carcinoma. Oncol Rep. 2006; 15:1507-11.
10. Ho J C, Ip Y C, Cheung S T, Lee Y T, Chan K F, Wong S Y, et al. Granulin-epithelin precursor as a therapeutic target for hepatocellular carcinoma. Hepatology. 2008; 47:1524-32.
11. Cheung P F, Cheng C K, Wong N C, Ho J C, Yip C W, Lui V C, et al. Granulin-epithelin precursor is an oncofetal protein defining hepatic cancer stem cells. PLoS One. 2011; 6:e28246.
12. Reya T, Morrison S J, Clarke M F, Weissman I L. Stem cells, cancer, and cancer stem cells. Nature. 2001; 414:105-11.
13. Hanahan D, Weinberg R A. Hallmarks of cancer: the next generation. Cell. 2011; 144:646-74.
14. Schatton T, Frank M H. Antitumor immunity and cancer stem cells. Ann N Y Acad Sci. 2009; 1176:154-69.
15. Noh K H, Kim B W, Song K H, Cho H, Lee Y H, Kim J H, et al. Nanog signaling in cancer promotes stem-like phenotype and immune evasion. J Clin Invest. 2012; 122:4077-93.
16. Schatton T, Schutte U, Frank N Y, Zhan Q, Hoerning A, Robles S C, et al. Modulation of T-cell activation by malignant melanoma initiating cells. Cancer Res. 2010; 70:697-708.
17. Gorczynski R M, Chen Z, Hu J, Kai Y, Lei J. Evidence of a role for C D200 in regulation of immune rejection of leukaemic tumour cells in C57B L/6 mice. Clin Exp Immunol. 2001; 126:220-9.
18. Siva A, Xin H, Qin F, Oltean D, Bowdish K S, Kretz-Rommel A. Immune modulation by melanoma and ovarian tumor cells through expression of the immunosuppressive molecule C D200. Cancer Immunol Immunother. 2008; 57:987-96.
19. Ma S, Tang K H, Chan Y P, Lee T K, Kwan P S, Castilho A, et al. miR-130b Promotes CD133(+) liver tumor-initiating cell growth and self-renewal via tumor protein 53-induced nuclear protein 1. Cell stem cell. 2010; 7:694-707.
20. Yang Z F, Ho D W, Ng M N, Lau C K, Yu W C, Ngai P, et al. Significance of C D90+ cancer stem cells in human liver cancer. Cancer Cell. 2008; 13:153-66.
21. Kumar V, Yi Lo P H, Sawai H, Kato N, Takahashi A, Deng Z, et al. Soluble MICA and a MICA Variation as Possible Prognostic Biomarkers for HBV-Induced Hepatocellular Carcinoma. PLoS One. 2012; 7:e44743.
22. Bauer S, Groh V, Wu J, Steinle A, Phillips J H, Lanier L L, et al. Activation of N K cells and T cells by NKG2D, a receptor for stress-inducible MICA. Science. 1999; 285:727-9.
23. Salih H R, Rammensee H G, Steinle A. Cutting edge: down-regulation of MICA on human tumors by proteolytic shedding. J Immunol. 2002; 169:4098-102.
24. Li J J, Pan K, Gu M F, Chen M S, Zhao J J, Wang H, et al. Prognostic value of soluble MICA levels in the serum of patients with advanced hepatocellular carcinoma. Chin J Cancer. 2012; 32:141-8.
25. Kohga K, Takehara T, Tatsumi T, Ohkawa K, Miyagi T, Hiramatsu N, et al. Serum levels of soluble major histocompatibility complex (MHC) class I-related chain A in patients with chronic liver diseases and changes during transcatheter arterial embolization for hepatocellular carcinoma. Cancer Sci. 2008; 99:1643-9.
26. Kohga K, Takehara T, Tatsumi T, Miyagi T, Ishida H, Ohkawa K, et al. Anticancer chemotherapy inhibits MHC class I-related chain a ectodomain shedding by downregulating ADAM10 expression in hepatocellular carcinoma. Cancer Res. 2009; 69:8050-7.
27. Groh V, Wu J, Yee C, Spies T. Tumour-derived soluble MIC ligands impair expression of NKG2D and T-cell activation. Nature. 2002; 419:734-8.
28. Sun D, Wang X, Zhang H, Deng L, Zhang Y. MMP9 mediates MICA shedding in human osteosarcomas. Cell Biol Int. 2011; 35:569-74.
29. Liu G, Atteridge C L, Wang X, Lundgren A D, Wu J D. The membrane type matrix metalloproteinase MMP14 mediates constitutive shedding of MHC class I chain-related molecule A independent of A disintegrin and metalloproteinases. J Immunol. 2010; 184:3346-50.
30. Liu Y, Xi L, Liao G, Wang W, Tian X, Wang B, et al. Inhibition of PC cell-derived growth factor (PCDGF)/granulin-epithelin precursor (GEP) decreased cell proliferation and invasion through downregulation of cyclin D and CDK4 and inactivation of MMP-2. BMC Cancer. 2007; 7:22.
31. Toth M, Chvyrkova I, Bernardo M M, Hernandez-Barrantes S, Fridman R. Pro-MMP-9 activation by the MT1-MMP/MMP-2 axis and MMP-3: role of TIMP-2 and plasma membranes. Biochem Biophys Res Commun. 2003; 308:386-95.
32. Cosman D, Mullberg J, Sutherland C L, Chin W, Armitage R, Fanslow W, et al. ULBPs, novel MHC class I-related molecules, bind to CMV glycoprotein UL16 and stimulate NK cytotoxicity through the NKG2D receptor. Immunity. 2001; 14:123-33.
33. Kamimura H, Yamagiwa S, Tsuchiya A, Takamura M, Matsuda Y, Ohkoshi S, et al. Reduced NKG2D ligand expression in hepatocellular carcinoma correlates with early recurrence. J Hepatol. 2012; 56:381-8.
34. Malmberg K J, Levitsky V, Norell H, de Matos C T, Carlsten M, Schedvins K, et al. IFN-gamma protects short-term ovarian carcinoma cell lines from CTL lysis via a CD94/NKG2A-dependent mechanism. J Clin Invest. 2002; 110:1515-23.
35. Speiser D E, Pittet M J, Valmori D, Dunbar R, Rimoldi D, Lienard D, et al. In vivo expression of natural killer cell inhibitory receptors by human melanoma-specific cytolytic T lymphocytes. J Exp Med. 1999; 190:775-82.
36. Chen A, Shen Y, Xia M, Xu L, Pan N, Yin Y, et al. Expression of the nonclassical HLA class I and MICA/B molecules in human hepatocellular carcinoma. Neoplasma. 2011; 58:371-6.
37. Zhang J, Pan L, Chen L, Feng X, Zhou L, Zheng S. Non-classical MHC-Iota genes in chronic hepatitis B and hepatocellular carcinoma. Immunogenetics. 2012; 64:251-8.

We claim:

1. A method of treating liver cancer, comprising administering to a subject that has liver cancer an effective amount of an agent that promotes natural killer (NK) cell activity.

2. The method according to claim 1, wherein the agent inhibits the activity of granulin-epithelin precursor (GEP).

3. The method according to claim 2, wherein the agent that inhibits the activity of GEP is an anti-GEP antibody.

4. The method according to claim 3, wherein the anti-GEP antibody is a monoclonal antibody.

5. The method according to claim 3, wherein the anti-GEP antibody is a polyclonal antibody.

6. The method of claim 2, wherein the agent is a siRNA or shRNA against GEP mRNA.

7. The method according to claim 1, comprising administering an effective amount of NK cells.

8. The method according to claim 1, wherein the agent that promotes NK cell activity is selected from IL-15, IL-12, pectin, IL-2, IFN-gamma, and IL-21.

9. The method according to claim 1, wherein the subject is a human.

10. A method of preventing recurrence of or treating liver cancer in a subject, wherein the method comprises administering to a subject an effective amount of an agent that inhibits the expression or activity of granulin-epithelin precursor (GEP), wherein the subject had surgical resection of a primary liver tumor, underwent hepatectomy, and/or had liver transplantation.

11. The method according to claim 10, comprising administering an agent that inhibits the activity of GEP.

12. The method according to claim 10, wherein the agent that inhibits the activity of GEP is an anti-GEP antibody.

13. The method according to claim 10, wherein the anti-GEP antibody is a monoclonal antibody.

14. The method according to claim 10, wherein the anti-GEP antibody is a polyclonal antibody.

15. The method according to claim 10, further comprising administering an effective amount of NK cells.

16. The method according to claim 10, further comprising administering an effective amount of an agent that promotes NK cell activity.

17. The method according to claim 16, wherein the agent that promotes NK cell activity is selected from IL-15, IL-12, pectin, IFN-gamma, IL-2, and IL-21.

18. The method according to claim 10, wherein the subject is a human.

19. A method of treating liver cancer, comprising administering to a subject that has liver cancer an effective amount of an agent that increases MHC class I chain-related molecule A (MICA) expression on liver cancer cells.

20. The method according to claim 19, wherein the agent inhibits the activity of granulin-epithelin precursor (GEP).
